(19) United States
(12) Patent Application Publication
(10) Pub. No.: US 2025/0064391 A1
(43) Pub. Date: Feb. 27, 2025

(54) SELECTIVE INCLUSION OF IMPEDANCE IN DEVICE-BASED DETECTION OF SLEEP APNEA

(71) Applicant: Medtronic, Inc., Minneapolis, MN (US)

(72) Inventors: Gautham Rajagopal, Minneapolis, MN (US); Yong K. Cho, Excelsior, MN (US); Shantanu Sarkar, Roseville, MN (US); Catherine R. Condie, Shoreview, MN (US); Sean R. Landman, Minneapolis, MN (US); Leonardo Rapallini, Edina, MN (US); Paul G. Krause, Mahtomedi, MN (US)

(21) Appl. No.: 18/948,243

(22) Filed: Nov. 14, 2024

Related U.S. Application Data
(63) Continuation-in-part of application No. PCT/IB2023/055586, filed on May 31, 2023.
(60) Provisional application No. 63/366,046, filed on Jun. 8, 2022.

(51) Int. Cl. A61B 5/00; A61B 5/0205; A61B 5/024; A61B 5/0245; A61B 5/0531; A61B 5/361

(52) U.S. Cl. CPC A61B 5/4818; A61B 5/0205; A61B 5/0531; A61B 5/361; A61B 5/02405; A61B 5/0245

(57) ABSTRACT

A medical device may be configured to determine heart rates of the patient based on the cardiac signal sensed during a first period. The medical device may detect one or more sleep apnea episodes of the patient occurring during the first period. The medical device may determine whether one or more verification conditions are satisfied. Responsive to determining that the one or more verification conditions are satisfied, the medical device may measure impedances of the patient during a second period subsequent to the first period, and use impedance to the measured impedances to detect one or more sleep apnea episodes of the patient occurring during the second period.

This application is a continuation-in-part of WO International Patent Application No. PCT/IB2023/055586, filed 31 May 2023, which claims the benefit of U.S. Provisional Patent Application Ser. No. 63/366,046, filed 8 Jun. 2022, the entire content of each application is incorporated herein by reference.

### TECHNICAL FIELD

This disclosure relates to techniques for detecting a sleep apnea episode.

### BACKGROUND

When functioning properly, a heart maintains its own intrinsic rhythm, and is capable of pumping adequate blood throughout a circulatory system. This intrinsic rhythm is a function of intrinsic signals generated by the sinoatrial (SA) node located in the upper right atrium. The SA node periodically depolarizes, which in turn causes the atrial heart tissue to depolarize so that right and left atria contract as the depolarization travels through the atrial heart tissue. The atrial depolarization signal is also received by the atrioventricular node, or AV node, which, in turn, triggers a subsequent ventricular depolarization signal that travels through and depolarizes the ventricular heart tissue causing the right and left ventricles to contract.

A condition known as sleep apnea can diminish cardiac output and pose various risks to patients, particularly those who are susceptible to heart failure. Sleep apnea is a sleep disorder that involves the temporary cessation of respiratory airflow during sleep. In various scenarios, sleep apnea may be characterized by one or both of pauses in breathing or periods of shallow breathing during sleep. Sleep apnea is generally considered a medical syndrome that occurs in various forms. One recognized form of sleep apnea is “central sleep apnea,” which is associated with a failure of the central nervous system to automatically initiate and control respiration. Another recognized form of sleep apnea is “obstructive sleep apnea,” which is associated with an obstruction of the airways due to airway collapse. Yet another recognized form of sleep apnea is a mixed form that may include a central nervous system failure to drive ventilatory effort combined with an obstructive apnea.

Possible effects of sleep apnea include daytime sleepiness, impaired alertness, and various associated cardiovascular diseases, which in turn can significantly impair patient lifestyle and increase morbidity risk. In some cases, obstructive sleep apnea can lead to death due to lack of oxygen to vital organs of the body. Various approaches have been taken to detect and treat sleep apnea.

### SUMMARY

Some medical device systems use cardiac signals to detect sleep apnea episodes. However, these medical device systems may have a non-negligible false positive error rate, particularly in specific cases. For instance, a medical device system that detects sleep apnea episodes based on heart rate may produce a higher rate of false-positives than normal when the patient is in atrial fibrillation (AF), when the patient has low heart rate variability due to certain medications such as beta blockers, during periodic limb movements, etc.

In accordance with techniques of this disclosure, a medical device system may detect one or more sleep apnea episodes of a patient based on the measured impedances in response to determining whether one or more verification conditions are satisfied. The one or more verification conditions may be associated with conditions under which detecting sleep apnea episodes based on heart rate may produce a higher rate of false-positives than normal. In some examples, the medical device system may analyze impedance measurements to detect respirations and, based on the respirations, detect sleep apnea. Hence, the medical device system may use impedance measurements to independently detect sleep apnea episodes and/or confirm sleep apnea episodes detected based on heart rate.

The techniques of this disclosure may provide one or more technical advantages that improve the operation of a medical device. For example, selective using impedance measurements to detect sleep apnea episodes may reduce the rate of false-positive sleep apnea episode detections. Further, selective use of impedance measurements to detect sleep apnea episodes may reduce consumption of limited power and other resources of a medical device relative to non-selective use of impedance measurements for primary detection of sleep apnea episodes.

In some examples, a medical device comprises: one or more electrodes; sensing circuitry configured to: sense a cardiac signal indicating activity of a heart of a patient; and measure impedances of the patient via the one or more electrodes; and processing circuitry configured to: determine heart rates of the patient based on the cardiac signal sensed during a first period; detect one or more sleep apnea episodes of the patient occurring during the first period based on the heart rates; determine whether one or more verification conditions are satisfied; and responsive to determining that the one or more verification conditions are satisfied: control the sensing circuitry to measure impedances of the patient during a second period subsequent to the first period; and detect one or more sleep apnea episodes of the patient occurring during the second period based on the measured impedances.

In some examples, a method comprises: sensing, by sensing circuitry of a medical device, a cardiac signal indicating activity of a heart of a patient; determining, by processing circuitry of the medical device, heart rates of the patient based on the cardiac signal sensed during a first period; detecting, by the processing circuitry, one or more sleep apnea episodes of the patient occurring during the first period based on the heart rates; determining, by the processing circuitry, whether one or more verification conditions are satisfied; and responsive to determining that the one or more verification conditions are satisfied: controlling, by the processing circuitry, the sensing circuitry to measure impedances of the patient during a second period subsequent to the first period; and determining whether one or more sleep apnea episodes of the patient occurred during the second period based on the measured impedances.

The details of one or more examples of this disclosure are set forth in the accompanying drawings and in the description below. Other features, objects, and advantages will be apparent from the description and drawings, and from the claims.

### BRIEF DESCRIPTION OF DRAWINGS

FIG. 1 is a conceptual diagram illustrating an example medical device system configured to detect sleep apnea in accordance with techniques of this disclosure.

FIG. 2 is a conceptual drawing illustrating an example configuration of a leadless implantable medical device of a medical device system.

FIG. 3 is a conceptual diagram illustrating another example medical device system configured to detect sleep apnea in accordance with techniques of this disclosure.

FIG. 4 is a block diagram illustrating an example medical device configured to sense cardiac electrogram and impedance signals of a patient for detection of sleep apnea in accordance with techniques of this disclosure.

FIG. 5 is a graph that illustrates sleep disordered heart rate (SDHR) information for a patient.

FIG. 6 is a graph that illustrates cardiac electrogram signals indicative of atrial fibrillation.

FIG. 7 is a graph that illustrates cardiac electrogram signals indicative of low heart variability.

FIG. 8 is a graph that illustrates cardiac electrogram signals that may be analyzed for limb movement in accordance with techniques of this disclosure.

FIG. 9 is a flowchart illustrating a method by which a medical device system that is configured to detect a sleep apnea episode in accordance with techniques of this disclosure.

FIG. 10 is a flowchart illustrating a method by which a medical device system that is configured to detect a sleep apnea episode in accordance with techniques of this disclosure.

FIG. 11 is a flowchart illustrating a method by which a medical device system that is configured to detect a sleep apnea episode in accordance with techniques of this disclosure.

### DETAILED DESCRIPTION

In general, this disclosure is directed to detecting individual episodes of sleep apnea. As used herein, “detection” refers to techniques for identifying an episode and does not necessarily imply that an episode has been identified. Accordingly, it is possible for a medical device system to initiate detection of sleep apnea episodes for a period of time and not detect any sleep apnea episodes (e.g., because no sleep apnea episodes occurred during the period of time).

Sleep apnea is a breathing disorder that cuts oxygen supply to various systems and organs of the body. To deal with the reduction in oxygenation levels, organs and systems of the body may trigger one or more compensatory mechanisms. With respect to the cardiovascular system, the compensatory mechanism(s) cause the heart to increase blood output for a period of time. As such, the cardiac compensatory mechanisms cause increased exertion of the heart. Moreover, at the end of a sleep apnea episode and during a recovery period that follows a sleep apnea episode, the patient's heart rate may increase significantly, due to alveolar hyperventilation caused by the pulmonary system's compensatory mechanisms. The heart rate spike after a sleep apnea episode may be greater in magnitude naturally occurring heart rate increases that are exhibited by the normal phenomenon of cyclical variation of heart rate (CVHR). As such, both the reduced oxygen supply during a sleep apnea episode and the hyperventilation that follows a sleep apnea episode may cause exertion levels in the heart that exceed normal levels of heart exertion.

The abnormal oxygenation conditions associated with sleep apnea may affect various systems and vital organs adversely. Repeated instances of increased heart exertion, as may be caused by frequent compensatory blood output to counter chronic sleep apnea and by increasing the heart rate to accommodate subsequent hyperventilation, increases the likelihood of heart ailments or possible heart failure. Some existing medical device systems have relied on respiratory measurement data to detect an episode of sleep apnea. For example, some medical device systems use heart rate to detect sleep apnea episodes. However, these medical device systems may be inaccurate (e.g., have a significant false positive error rate) in certain cases, which may lead to inadequate treatment of sleep apnea.

A medical device system may use heart rate to detect sleep apnea episodes of a patient. For example, the medical device system may detect sleep apnea episodes based on one or more heart rate parameters. Examples of heart rate parameters may include heart rate variability (HRV) values and other parameters that may be derived from HRV. Although generally effective, a medical device system that uses heart rate to detect sleep apnea episodes may be less accurate in certain cases. For instance, a medical device system that uses heart rate to detect sleep apnea episodes may produce a higher rate of false-positives than normal when the patient is in atrial fibrillation (AF), when the patient has low heart rate variability due to certain medications such as beta blockers, during periodic limb movements, etc.

In accordance with techniques of this disclosure, a medical device system may use impedance to detect sleep apnea episodes in response to determining whether one or more verification conditions are satisfied. The one or more verification conditions may be associated with conditions under which a medical device system that uses heart rate to detect sleep apnea episodes may produce a higher rate of false-positives than normal. In some examples, a medical device system that uses impedance to detect sleep apnea episodes may analyze impedance measurements to detect respirations and, based on the respirations, detect sleep apnea. Hence, a medical device system may use impedance measurements to independently detect sleep apnea episodes and/or confirm sleep apnea episodes detected based on heart rate.

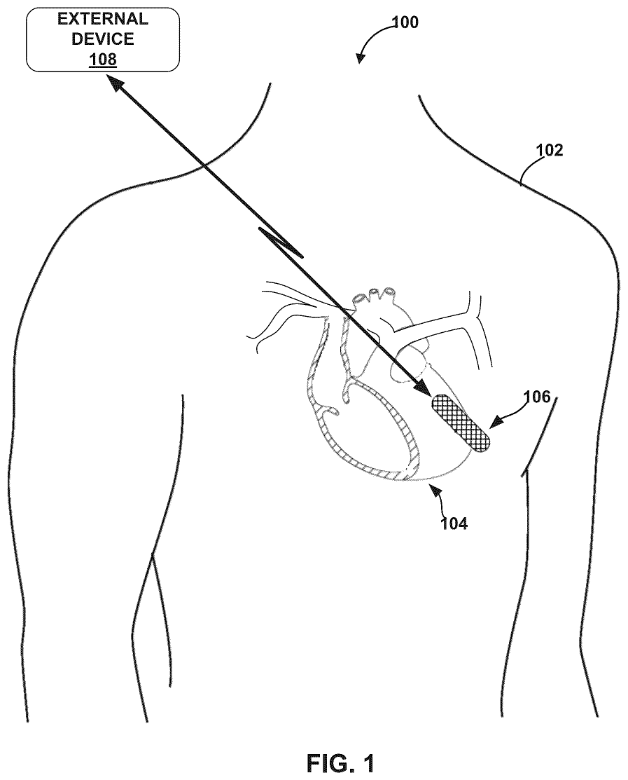

FIG. 1 is a conceptual diagram illustrating a medical device system 100 in conjunction with a patient 102. Medical device system 100 is an example of medical device system configured to implement the techniques described herein for detecting a sleep apnea episode. In the example of FIG. 1, medical device system 100 may include a medical device 106 and an external device 108. Medical device 106 may be an implantable medical device (IMD), such as an insertable cardiac monitor (ICM), an implantable pacemaker, implantable defibrillator, implantable neurostimulator, etc. In some examples, although described primarily as implantable, medical device 106 is an external medical device, such as a patch, watch, headband, or other wearable medical device.

Medical device 106 is capable of sensing and recording cardiac electrogram (EGM) signals from a position outside of heart 104 via electrodes of the medical device. In some examples, medical device 106 may include or be coupled to one or more additional sensors and/or sense one or more other physiological signals, such as signals that vary based on heart motion and/or sounds, blood pressure, blood flow, blood oxygenation, or respiration. For example, medical device 106 may be capable of measuring impedance via the electrodes, and generating an impedance signal that varies based on respiration based on the measured impedances. Medical device 106 may be implanted outside of the thorax of patient, e.g., subcutaneously or submuscularly, such as the pectoral location. In some examples, medical device 106 may take the form of a Reveal LINQ™ ICM.

External device 108 may be a computing device, e.g., used in a home, ambulatory, clinic, or hospital setting, to communicate with medical device 106 via wireless telemetry. External device 108 may be coupled to a remote patient monitoring system, such as Carelink®. External device 108 may be, for example, a programmer, external monitor, or consumer device (e.g., smart phone). External device 108 may be used to program commands or operating parameters into medical device 106 for controlling its functioning, e.g., when configured as a programmer for medical device 106. External device 108 may be used to interrogate medical device 106 to retrieve data, including device operational data as well as physiological data accumulated in memory, such as quantifications of detected sleep apnea episodes. The interrogation may be automatic, e.g., according to a schedule, or in response to a remote or local user command. Examples of communication techniques used by medical device 106 and external device 108 may include tissue conductance communication (TCC), or radiofrequency (RF) telemetry, which may be an RF link established via Bluetooth®, WiFi®, or medical implant communication service (MICS).

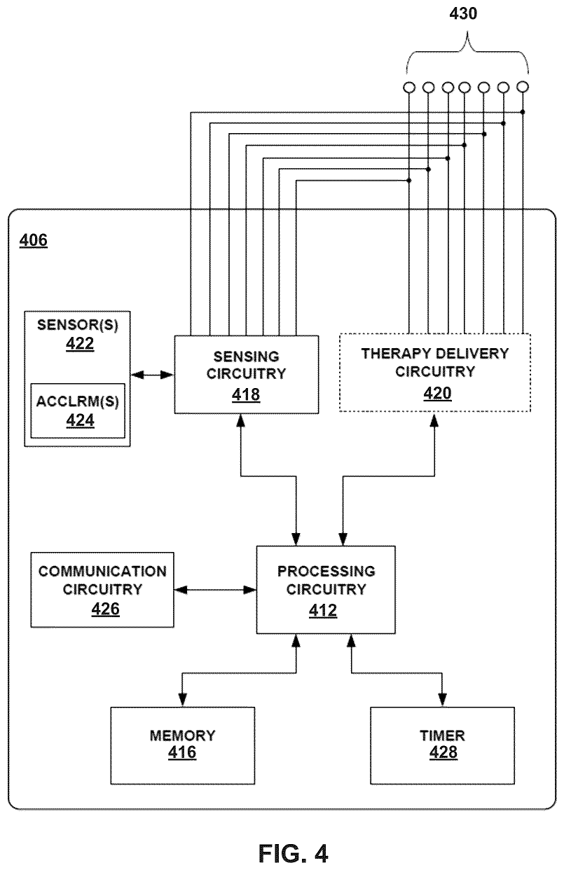

Both medical device 106 and external device 108 may include processing circuitry, and the processing circuitry of either device, of both devices, or any other device included in medical device system 100 may perform the techniques described herein. Based on analysis of patient's heart activity and/or impedance in accordance with techniques of this disclosure, the processing circuitry of one or more of the devices (e.g., IMD 106 and/or external device 108 may also be configured to provide an indication to a user, e.g., to a clinician and/or to patient 102 that a likely sleep apnea episode has been detected, e.g., via external device 108. For example, medical device system 100 may store (e.g., record) data associated with the occurrence of the likely sleep apnea episode in memory (e.g., as illustrated in FIG. 4) included in medical device system 100 (e.g., memory of either medical device 106 or external device 108, or any other memory included in medical device system 100, such as memory included in a server, etc.). Medical device system 100 may then present the data (e.g., as part of a report or history of patient 102) to patient 102, a clinician or caregiver of patient 102, or other user. Patient 102, a clinician, a caregiver or another implanted or external medical device may use the data to deliver or take a preventative measure to prevent the occurrence of additional sleep apnea episodes.

Although not illustrated in the example of FIG. 1, a medical device system configured to implement the techniques described in this disclosure may include one or more implanted or external medical devices in addition to or instead of medical device 106. For example, a medical device system may include a vascular ICD, an extravascular ICD, a cardiac pacemaker implanted outside of the heart 104 but coupled to intracardiac or epicardial leads, or an intracardiac pacing device. One or more such devices may generate signals, and include processing circuitry configured to perform, in whole or in part, the techniques described herein for detecting sleep apnea episode(s). The implanted devices may communicate with each other and/or an external device 108, and one of the implanted or external devices may ultimately determine whether sleep apnea is detected based on information received from the other device(s).

In various examples, implantable medical device (IMD) components may be connected to leads that extend into heart 104 or could be implanted in heart 104 entirely. In some examples, components of medical device system 100 may be external devices. Components of medical device system 100 may be configured to detect cardiac electrogram signals, such as an ECG. In various examples, processing circuitry of medical device system 100, such as processing circuitry of medical device 106 and/or external device 108, may perform the techniques of this disclosure using various types of sensing circuitry, such as sensing circuitry of medical device 106. For instance, the sensing circuitry may be configured to sense a cardiac signal indicating activity of heart 104 of patient 102. Medical device 106 may include electrodes configured to sense electrical signals associated with patient state. The electrodes may be coupled to the sensing circuitry. In such examples, the sensing circuitry may be configured to, e.g., measure impedances of patient 102 via the plurality of electrodes.

In general, detecting sleep apnea episodes based on heart rate (which may include measuring and analyzing heart rates) may require less power (e.g., consume less energy stored in a battery of medical device system 100) than detecting sleep apnea episodes based on impedance (which may include measuring and analyzing impedances). Accordingly, detecting sleep apnea episodes based on heart rate instead of based on impedance may conserve power of medical device system 100. However, as noted above, detecting sleep apnea episodes based on heart rate may be less accurate than normal in specific cases, such as when a patient is in AF, when the patient has low heart rate variability due to certain medications such as beta blockers, during periodic limb movements, etc. Hence, in accordance with techniques of this disclosure, medical device system 100 may be configured to use impedance in situations where detecting sleep apnea episodes based on heart rate may produce a higher rate of false-positives than normal. In this way, medical device system 100 may use impedance measurements to independently detect sleep apnea episodes and/or confirm sleep apnea episodes detected based on heart rate.

Processing circuitry of medical device system 100 may be configured to determine heart rates of patient 102 based on a cardiac signal sensed during a first period of time. The processing circuitry may use heart rate (described in greater detail below) to the determined heart rates to detect one or more sleep apnea episodes of the patient occurring during the first period. In some examples, the processing circuitry may determine one or more heart rate parameters (discussed in greater detail below) based on the determined heart rates of patient 102. The processing circuitry may detect sleep apnea episodes of patient 102 occurring during the first period based on the one or more heart rate parameters.

It should be understood that in some examples the processing circuitry may detect sleep apnea episodes occurring during the first period after the first period. That is, in some examples, sensing circuitry may sense the cardiac signal during the first period, and the processing circuitry may analyze the cardiac signal for the first period after the first period to detect whether one or more sleep apnea episodes occurred during the first period.

The processing circuitry may be configured to determine whether one or more verification conditions are satisfied. The verification conditions may be associated with conditions under which detecting sleep apnea episodes based on heart rate may produce a higher rate of false-positives than normal. Example verification conditions may include, but are not limited to, detection of one or more AF episodes during the first period, a threshold number (e.g., 5, 10, etc.) and/or a threshold rate (e.g., 1 sleep apnea episode per 2 minutes) of sleep apnea episodes occurring during the first period, receipt of an input indicating that patient 102 has a prescription associated with a low heart rate variability (e.g., beta-adrenoceptor blocking agents), one or more HRV values of patient 102 being less than a HRV threshold (e.g., 40 milliseconds (ms), 50 ms, 60 ms, etc.), and user input (e.g., a command inputted by a clinician for medical device system 100 to detect sleep apnea episodes based on measured impedances for a period of time (e.g., 2 hours, 6 hours, etc.)).

Responsive to determining that the one or more verification conditions are satisfied, the processing circuitry may control the sensing circuitry to measure impedance during a second period of time. The processing circuitry may then detect one or more sleep apnea episodes during the second period of time based on the measured impedances. The processing circuitry may detect respirations based on the measured impedances of patient 102. For instance, when patient 102 inhales, measured impedance may increase and reach its highest value at the end of inhalation. Conversely, when patient 102 exhales, measured impedance may decrease and reach its lowest value at the end of exhalation.

The processing circuitry may identify a pattern of inhalation and exhalation and identify anomalies indicative of an absence of respiration when patient 102 is sleeping (i.e., a sleep apnea episode). Thus, the processing circuitry may detect the one or more sleep apnea episodes of patient 102 occurring during the second period based on the respirations (or absence thereof) of patient 102. In some examples, responsive to the processing circuitry detecting one or more sleep apnea episodes of patient 102 occurring during the second period, the processing circuitry may output a report indicating information about the detected sleep apnea episodes (e.g., the time of day the episodes occurred, the number of episodes, the duration of each episode, etc.).

It should be understood that in some examples the processing circuitry may detect sleep apnea episodes occurring during the second period after the second period. That is, in some examples, sensing circuitry may measure impedance during the second period, and the processing circuitry may analyze the impedances measured during the second period after the second period to detect whether one or more sleep apnea episodes occurred during the second period.

The second period of time (e.g., when the sensing circuitry is measuring impedance) may be subsequent to the first period of time (e.g., when the sensing circuitry is sensing cardiac signals). The duration of the second period may be predetermined, such that the sensing circuitry stops measuring impedances in response to the second period lasting for the predetermined duration. An example predetermined duration of the second period is two minutes.

Selectively detecting sleep apnea episodes based on impedance may advantageously conserve power of medical device 106. This is because although detection of sleep apnea episodes based on impedance may be more accurate than the detection of sleep apnea episodes based on heart rate (at least in certain scenarios), measuring and analyzing impedance may consume more power than measuring and analyzing cardiac activity. At a minimum, measuring and analyzing both cardiac activity and impedance may consume more power than just measuring and analyzing cardiac activity (the latter of which may be highly useful not only for the detection of sleep apnea episodes but also other purposes). Therefore, the techniques of this disclosure enable selectively detecting sleep apnea episodes based on impedance during conditions when detecting sleep apnea episodes based on heart rate may be less accurate and stopping detection of sleep apnea episodes based on impedance when those conditions no longer exist. In turn, the techniques of this disclosure may improve detection of sleep apnea episodes without substantially increasing power consumption.

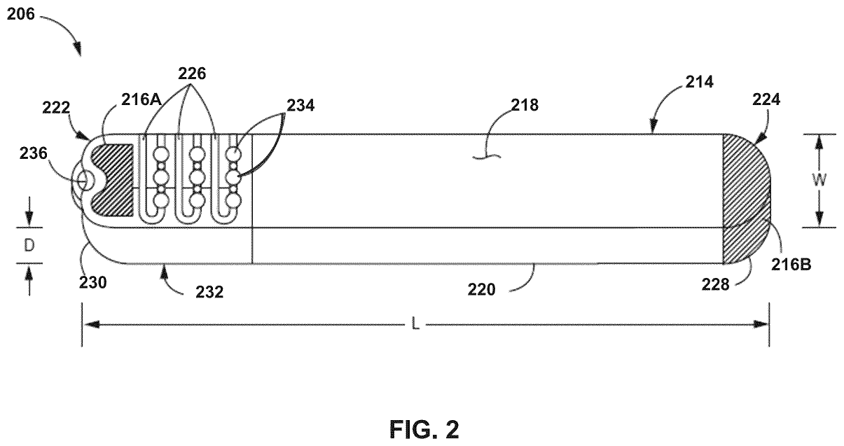

FIG. 2 is a conceptual drawing illustrating an example configuration of an IMD 206. IMD 206 may be substantially similar to IMD 106 of FIG. 1. In the example shown in FIG. 2, IMD 206 may comprise a leadless, subcutaneously-implantable monitoring device having a housing 214, a proximal electrode 216A, and a distal electrode 216B. Housing 214 encloses electronic circuitry located inside the IMD 206, and protects the circuitry contained therein from fluids such as body fluids. In some examples, housing 214 may comprise first major surface 218, second major surface 220, proximal end 222, and distal end 224. Proximal electrode 216A and distal electrode 216B may be positioned near respective proximal and distal ends 222 and 224 of IMD 206, such that a spacing between proximal electrode 216A and distal electrode 216B may range from about 30-55 mm, about 35-55 mm, or about 40-55 mm, or more generally from about 25-60 mm. In some examples, IMD 206 may include one or more additional electrodes and/or one or more other sensors (not shown), which may be positioned on one or both of major surfaces 218, 220 of IMD 206. In any such examples, electrical feedthroughs may provide electrical connection of electrodes 216A, 216B or other sensors to circuitry within housing 214.

In the example shown in FIG. 2, IMD 206 is defined by a length L, a width W, and thickness or depth D. In this example, IMD 206 is in the form of an elongated rectangular prism in which length L is significantly greater than width W, and in which width W is greater than depth D. However, other configurations of IMD 206 are contemplated, such as those in which the relative proportions of length L, width W, and depth D vary from those described and shown in FIG. 2. In some examples, the geometry of the IMD 206, such as the width W being greater than the depth D, may be selected to allow IMD 206 to be inserted under the skin of the patient using a minimally invasive procedure and to remain in the desired orientation during insertion. In addition, IMD 206 may include radial asymmetries (e.g., the rectangular shape) along a longitudinal axis of IMD 206, which may help maintain the device in a desired orientation following implantation.

Overall, IMD 206 may have a length L of about 20-30 mm, about 40-60 mm, or about 45-60 mm. In some examples, the width W of first major surface 218 may range from about 3-10 mm, and may be any single width or range of widths between about 3-10 mm. In some examples, a depth D of IMD 206 may range from about 2-9 mm. In other examples, the depth D of IMD 206 may range from about 2-5 mm, and may be any single or range of depths from about 2-9 mm. In any such examples, IMD 206 is sufficiently compact to be implanted within the subcutaneous space of patient 102 in the region of a pectoral muscle.

IMD 206 may have a geometry and size designed for ease of implantation and patient comfort. For example, IMD 206 may have a volume of 3 cubic centimeters (cm³) or less, 1.5 cm³ or less, or any volume therebetween. As illustrated in FIG. 2, proximal end 222 and distal end 224 may be rounded, which may reduce discomfort and/or irritation to surrounding tissue when IMD 206 implanted under the skin of patient 102. An example configuration of IMD 206, including an instrument and method for inserting IMD 206 is described in U.S. Patent Publication No. 2014/0276928. An example configuration of IMD 206 also is described in U.S. Patent Publication No. 2016/0310031.

In some examples, IMD 206 may be configured for implantation within patient 102 such that first major surface 218 of IMD 206 faces outward towards the skin when IMD 206 is inserted within patient 102 and second major surface 220 is faces inward toward musculature of patient 102. First and second major surfaces 218, 220 may face in directions along a sagittal axis of patient 102, as illustrated in FIG. 1, and this orientation may be maintained upon implantation due to the dimensions of IMD 206. Additionally, or alternatively, IMD 206 may be configured for implantation within patient 102 in one or more other orientations relative to one or more anatomical landmarks of patient 102.

In the example shown in FIG. 2, proximal end 222 of IMD 206 includes header assembly 232 having one or more of integrated antenna 226, anti-migration projections 234, and suture hole 236. Integrated antenna 226 is located on the same major surface (e.g., first major surface 218) as electrode 216A, and may be an integral part of header assembly 232. In other examples, integrated antenna 226 may be formed on the major surface opposite from electrode 216A or may be incorporated within housing 214 of IMD 206. Antenna 226 may be configured to transmit or receive electromagnetic signals for communication. For example, antenna 226 may be configured to transmit to or receive signals from a programmer via inductive coupling, electromagnetic coupling, tissue conductance, Near Field Communication (NFC), Radio Frequency Identification (RFID), Bluetooth, WiFi, or other proprietary or non-proprietary wireless telemetry communication schemes. Antenna 226 may be coupled to communication circuitry of IMD 206, which may drive antenna 226 to transmit signals to an external device (e.g., external device 108). Antenna 226 may transmit signals received from the external device to processing circuitry of IMD 206 via the communication circuitry.

IMD 206 may include several features for retaining IMD 206 in position once subcutaneously implanted in patient 102. For example, as shown in FIG. 2, housing 214 may include anti-migration projections 234 positioned adjacent integrated antenna 226. Anti-migration projections 234 may comprise a plurality of bumps or protrusions extending away from first major surface 218 and may reduce or prevent movement of IMD 206 after implantation in patient 102. In other examples, anti-migration projections 234 may be located on the opposite major surface as proximal electrode 216A and/or integrated antenna 226. In addition to or instead of anti-migration projections 234, a portion of housing 214 (e.g., header assembly 232) may define a suture hole 236, which may enable a clinician to suture IMD 206 to patient tissue to reduce or prevent movement of IMD 206 after implantation in patient 102. In the example of FIG. 2, suture hole 236 is defined by a portion of header assembly 232 adjacent to proximal electrode 216A. In some examples, header assembly 232 may comprise a molded header assembly made from a polymeric material, which may be integral with or separable from the main portion of IMD 206.

In examples in which the processing circuitry of the medical device system including IMD 206 is configured to measure heart activity based on a cardiac electrogram signal, IMD 206 may include a plurality of electrodes. For example, as illustrated in FIG. 2, IMD 206 may include a proximal electrode 216A and a distal electrode 216B. As shown in the illustrated example, proximal electrode 216A may be positioned on header assembly 232, and distal electrode 216B may be formed from an uninsulated distal portion of conductive housing 214. Proximal electrode 216A and distal electrode 216B may be positioned near respective proximal and distal ends 222 and 224 of IMD 10, such that a spacing between proximal electrode 216A and distal electrode 216B may range from about 30-55 mm, about 35-55 mm, or about 40-55 mm, or more generally from about 25-60 mm. In some examples, IMD 206 also may include one or more additional electrodes (not shown) positioned on one or both of major surfaces 218, 220 of IMD 10. In any such examples, electrical feedthroughs may provide electrical connection of electrodes 216A, 216B, any additional electrodes, and antenna 226, to circuitry within housing 214.

In the example shown in FIG. 2, proximal electrode 216A is in close proximity to proximal end 222, and distal electrode 216B is in close proximity to distal end 224 of IMD 10. In this example, distal electrode 216B is not limited to a flattened, outward facing surface, but may extend from first major surface 218, around rounded edges 228 or end surface 230, and onto the second major surface 220 in a three-dimensional curved configuration. As illustrated, proximal electrode 216A is located on first major surface 218 and is substantially flat and outward facing. However, in other examples not shown here, proximal electrode 216A and distal electrode 216B both may be configured like proximal electrode 216A shown in FIG. 2, or both may be configured like distal electrode 216B shown in FIG. 2. Any of electrodes 216A, 216B may be formed of a biocompatible conductive material. For example, any of electrodes 216A, 216B may be formed from any of stainless steel, titanium, platinum, iridium, or alloys thereof. In addition, electrodes of IMD 206 may be coated with a material such as titanium nitride or fractal titanium nitride, although other suitable materials and coatings for such electrodes may be used.

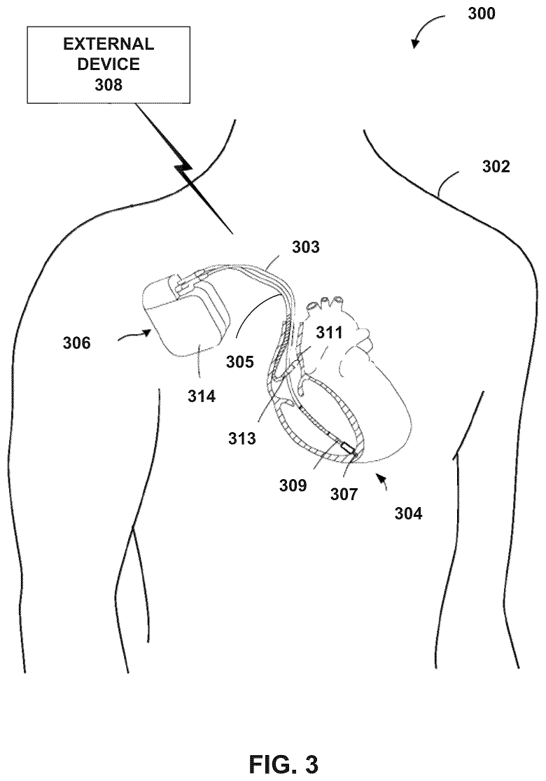

FIG. 3 is a conceptual drawing illustrating an example medical device system 300 in conjunction with a patient 302. Medical device system 300 may be substantially similar to medical device system 100 except for any differences described herein. Medical device system 100 and medical device system 300 are examples of medical device systems configured to implement the techniques described herein for detecting a sleep apnea episode. In the illustrated example, medical device system 300 includes a medical device 306 coupled to a ventricular lead 303 and an atrial lead 305. In some examples, upon identifying a likely sleep apnea episode, medical device system 300 may responsively provide an indication indicating that a sleep apnea episode is likely occurring, and/or deliver a therapy (e.g., using ventricular lead 303 and atrial lead 305) configured to prevent, mitigate, or remedy the effects of the sleep apnea episode.

Ventricular lead 303 and atrial lead 305 may be electrically coupled to medical device 306 and extend into the patient's heart 304. Ventricular lead 303 may include electrodes 307 and 309 shown positioned on the lead in the patient's right ventricle (RV) for sensing EGM signals and pacing in the RV. Atrial lead 305 may include electrodes 311 and 313 positioned on the lead in the patient's right atrium (RA) for sensing atrial EGM signals and pacing in the RA.

Medical device 306 may use both ventricular lead 303 and atrial lead 305 to acquire cardiac electrogram (EGM) signals from heart 304 of patient 102B. Medical device system 300 is shown as having a dual chamber IMD configuration, but other examples may include one or more additional leads, such as a coronary sinus lead extending into the right atrium, through the coronary sinus and into a cardiac vein to position electrodes along the left ventricle (LV) for sensing LV EGM signals and delivering pacing pulses to the LV. In other examples, a medical device system may be a single chamber system, or otherwise not include atrial lead 305.

Processing circuitry, sensing circuitry, and other circuitry configured for performing the techniques described herein may be housed within a sealed housing 314 of medical device 306. Housing 314 (or a portion thereof) may be conductive to serve as an electrode for pacing or sensing. Medical device 306 may acquire signal data (e.g., EGM signal data) and cardiac rhythm episode data and transmit the data to an external device 308. External device 308 may be a computing device, e.g., a device used in a home, ambulatory, clinic, or hospital setting, comprising processing circuitry and/or communicative interfacing circuitry configured to communicate with medical device 306 via wireless telemetry. External device 308 may be coupled to a remote patient monitoring system, such as Carelink®. External device 308 may include, be, or may be part of, in various examples, a programmer, external monitor, or consumer device, e.g., a smart phone.

External device 308 may be implemented and may operate in a manner similar to external device 108. For example, external device 308 may be used to program commands or operating parameters into medical device 306 for controlling its functioning, e.g., when configured as a programmer for medical device 306, and may be used to interrogate medical device 306 to retrieve data, including device operational data as well as physiological data accumulated in a memory of medical device 306. Medical device system 300 is an example of a medical device system operable to leverage physiological parameter value(s) to detect sleep apnea episodes. For example, medical device system 300 may be configured to monitor physiological parameter value(s) and determine whether one or more physiological parameter value satisfy one or more conditions. In some examples, if medical device system 300 determines that a sleep apnea episode has likely occurred, medical device system 300 may responsively provide an indication indicating that the event is detected, and optionally, trigger delivery of a therapy configured to remediate the effects of the event or stem the progression of such effects. The techniques may be performed by processing circuitry of medical device system 300, such as processing circuitry of one or both of medical device 306 and external device 308, individually, or collectively.

The processing circuitry of external device 308 and/or medical device 306 may determine the values of at least some patient parameters based on signals generated by sensing circuitry of medical device 306. In some examples, medical device 306 may include or be coupled to one or more other sensors that generate one or more other physiological signals, such as signals that vary based on blood flow and/or respiration. The processing circuitry of external device 308 and/or medical device 306 may determine patient parameters based on therapy delivered by various components of medical device system 300 that are omitted from FIG. 3 for ease of illustration, such as a CPAP machine. For instance, the processing circuitry of external device 308 and/or medical device 306 may analyze physiological parameters of patient 302 to determine whether a sleep apnea episode has been remediated effectively by way of therapy delivered by the positive airway pressure machine or medical device 306.

FIG. 4 is a functional block diagram illustrating an example configuration of a medical device 406. Medical device 306 may correspond to medical device 106 in FIG. 1, medical device 206 in FIG. 2, medical device 306 in FIG. 3, or another medical device configured to implement the techniques as described in this disclosure. Similarly, although not illustrated in FIG. 4, external device 408 may correspond to external device 108 in FIG. 1, external device 308 illustrated in FIG. 3, or another external device configured to implement the techniques as described in this disclosure.

In the illustrated example, medical device 406 includes processing circuitry 412, memory 416, sensing circuitry 418, therapy delivery circuitry 420, one or more sensors 422 (e.g., an accelerometer 424), communication circuitry 426, and timer 428. However, medical device 406 does not need to include all these components in some examples, or medical device 406 may include additional components in some examples.

Memory 416 may include computer-readable instructions that, when executed by processing circuitry 412, cause medical device 406 and processing circuitry 412 to perform various functions attributed to medical device 406 and processing circuitry 412 herein (e.g., determining time of day, comparing time of day to a predetermined window, causing communication circuitry 426 to transmit physiological parameter value(s) to external device, etc.). Memory 416 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital or analog media. Memory 416 may store threshold(s) for the peak-to-valley time interval condition, the activity count condition, the peak-to-valley heart rate variation condition, the cycle length condition, etc.

Processing circuitry 412 may include fixed function circuitry and/or programmable processing circuitry. Processing circuitry 412 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 412 may include multiple components, such as any combination of one or more microprocessors, one or more controllers, one or more DSPs, one or more ASICs, or one or more FPGAs, as well as other discrete or integrated logic circuitry. The functions attributed to processing circuitry 412 herein may be embodied as software, firmware, hardware or any combination thereof. For example, processing circuitry 412 may be processing circuitry of either medical device 406 or external device 408 or any other processing circuitry included in a medical device system may be configured to perform techniques in accordance with this disclosure.

Sensing circuitry 418 and therapy delivery circuitry 420 are coupled to electrodes 430. Electrodes 430 illustrated in FIG. 4 may correspond to, for example, electrodes carried on leads 303 and/or 305 of medical device system 300 (FIG. 3). Sensing circuitry 418 may monitor signals from a selected two or more of electrodes 430 in order to monitor electrical activity of heart, impedance, or other electrical phenomenon. For example, sensing circuitry 418 may be configured to, via electrodes 430, sense a cardiac signal indicating activity of heart 104 of patient 102, measure impedances of patient 102, etc. In some examples, sensing circuitry 418 may include one or more filters and amplifiers for filtering and amplifying a signal received from electrodes 430.

Sensing circuitry 418 may include a switch module to select which of the available electrodes 430 (or electrode polarities) are used to sense the heart activity. In examples with several electrodes 430, processing circuitry 412 may select the electrodes that function as sense electrodes, i.e., select the sensing configuration, via the switch module within sensing circuitry 418. Sensing circuitry 418 may also pass one or more digitized EGM signals to processing circuitry 412 for analysis, e.g., for use in cardiac activity discrimination (e.g., cardiac rhythm discrimination).

Processing circuitry 412 may determine physiological parameters or patient states based on measurements from sensing circuitry 418. For instance, processing circuitry 412 may determine heart rates, HRV, an impedance signal that varies based on respiration, etc., based on the cardiac signal and impedance measurements obtained by sensing circuitry 418 via electrodes 430. Processing circuitry 412 may also detect the occurrence of medical conditions, such as sleep apnea, arrhythmias (e.g., tachyarrhythmias or bradycardia), atrial fibrillation, based on the signals.

In the example of FIG. 4, medical device 406 includes one or more sensors 422 coupled to sensing circuitry 418. Although illustrated in FIG. 4 as being included within medical device 406, one or more of sensors 422 may be external to medical device 406, e.g., coupled to medical device 406 via one or more leads, or configured to wirelessly communicate with medical device 406. In some examples, sensors 422 transduce a signal indicative of a patient parameter, which may be amplified, filtered, or otherwise processed by sensing circuitry 418. In such examples, processing circuitry 412 determines physiological parameter value(s) based on the signals. In some examples, sensors 422 determine the physiological parameter value(s), and communicate them, e.g., via a wired or wireless connection, to processing circuitry.

In some examples, sensors 422 include one or more accelerometers 424, e.g., one or more three-axis accelerometers. Signals generated by the one or more accelerometers 424 may be indicative of, for example, heart sounds or other vibrations or movement associated with the beating of the heart, or coughing, rales, or other respiration abnormalities. Accelerometers 424 may produce and transmit signals to processing circuitry 412 for a determination as to whether the patient's heart has contracted. In some examples, sensors 422 may include one or more microphones configured to detect heart sounds or respiration abnormalities. In some examples, sensors 422 may include sensors configured to transduce signals indicative of blood flow, oxygen saturation of blood, or patient temperature, and processing circuitry may determine patient parameters values based on these signals. In some examples, sensors 422 may include optical sensors configured to detect optical signals, such as a photoplethysmography (PPG) signal. In some examples, sensors may include optical sensors configured to detect optical signals that indicate at least one of an amount or level of tissue oxygenation and/or an amount or level of blood oxygen.

Therapy delivery circuitry 420 is configured to generate and deliver electrical therapy to the heart. Therapy delivery circuitry 420 may include one or more pulse generators, capacitors, and/or other components capable of generating and/or storing energy to deliver as pacing therapy, defibrillation therapy, cardioversion therapy, other therapy or a combination of therapies. In some instances, therapy delivery circuitry 420 may include a first set of components configured to provide pacing therapy and a second set of components configured to provide anti-tachyarrhythmia shock therapy. In other instances, therapy delivery circuitry 420 may utilize the same set of components to provide both pacing and anti-tachyarrhythmia shock therapy. In still other instances, therapy delivery circuitry 420 may share some of the pacing and shock therapy components while using other components solely for pacing or shock delivery.

Therapy delivery circuitry 420 may include charging circuitry, one or more charge storage devices, such as one or more capacitors, and switching circuitry that controls when the capacitor(s) are discharged to electrodes 430 and the widths of pulses. Charging of capacitors to a programmed pulse amplitude and discharging of the capacitors for a programmed pulse width may be performed by therapy delivery circuitry 420 according to control signals received from processing circuitry, which are provided by processing circuitry according to parameters stored in memory 416. Processing circuitry 412 may control therapy delivery circuitry 420 to deliver the generated therapy to the heart via one or more combinations of electrodes 430, e.g., according to parameters stored in memory 416. Therapy delivery circuitry 420 may include switch circuitry to select which of the available electrodes 430 are used to deliver the therapy, e.g., as controlled by processing circuitry.

Communication circuitry 426 may include any suitable hardware, firmware, software or any combination thereof for communicating with another device, such as an external device 408 or another IMD or sensor. Under the control of processing circuitry, communication circuitry 426 may receive downlink telemetry from and send uplink telemetry to external device 408 or another device with the aid of an antenna, which may be internal and/or external. In some examples, communication circuitry 426 may communicate with a local external device, and processing circuitry may communicate with a networked computing device via the local external device and a computer network, such as the Medtronic CareLink® Network.

A clinician or other user may retrieve data from medical device 406 using external device 408 or another local or networked computing device configured to communicate with processing circuitry via communication circuitry 426. The clinician may also program parameters of medical device 406 using external device 408 or another local or networked computing device.

Although not illustrated in FIG. 4, communication circuitry 426 may be coupled to electrodes 430 for tissue conductance communication (TCC) via the electrodes. In some examples, communication with medical device 406 and external device 408 may be via RF telemetry or TCC.

Medical device 406 and/or external device 408 may include timer 420. Timer 420 may be configured to provide a timer value, which processing circuitry may use to measure and determine various physiological parameters that involve duration. Additionally or alternatively, processing circuitry 412 may determine a time of day based on the timer value. For example, processing circuitry 412 may be configured to determine a time of day based on the timer value. Processing circuitry 412 may associate heart activity of the patient with the time of day when the heart activity occurred. For example, processing circuitry may associate one or more sleep apnea episodes with the time of day when the sleep apnea episodes occurred and store this information in memory 416, which may be retrieved, for example, during interrogation by external device 408.

Processing circuitry 412 may be configured to determine physiological parameters and/or conditions of patient 102. For example, processing circuitry 412 may be configured to determine heart rates of patient 102 based on a cardiac signal (e.g., obtained by sensing circuitry 418), detect sleep apnea episodes based on heart rate and/or impedance, detect atrial fibrillation episodes based on a cardiac signal, determine HRV values based on determined heart rates, etc. Processing circuitry 412 may use one or more algorithms or methods to determine HRV values. Example methods may include the “Root Mean Square of Successive Differences” (RMSSD), the standard deviation of N-N intervals (SDNN), the standard deviation of sequential 5-minute N-N intervals (SDANN).

In some examples, processing circuitry 412 may be configured to determine a sleep apnea risk score based on one or more of determined physiological parameter value(s), frequency of detected sleep apnea events, duration of a sleep apnea event, presence of arrhythmia in patient 102, whether patient 102 is non-complaint with continuous positive airway pressure (CPAP), heart rate variability of patient 102, presence of AF in patient 102, risk of heart failure, or risk of chronic obstructive pulmonary disease (COPD) exacerbation. In some examples, processing circuitry 412 may be configured to apply one or more of determined physiological parameter value(s), frequency of detected sleep apnea events, duration of a sleep apnea event, presence of arrhythmia in patient 102, whether patient 102 is non-complaint with continuous positive airway pressure (CPAP), heart rate variability of patient 102, presence of AF in patient 102, risk of heart failure, or risk of chronic obstructive pulmonary disease (COPD) exacerbation to a machine learning model to determine a sleep apnea risk score.

Additionally, processing circuitry 412 may determine whether one or more verification conditions are satisfied based on the determined physiological parameters and/or conditions of patient 102. For example, responsive to detecting one or more AF episodes during detection of sleep apnea episodes based on heart rate (e.g., during a first period of time), processing circuitry 412 may determine that one or more verification conditions have been satisfied and determine whether one or more sleep apnea episodes occurred based on measured impedance. In another example, responsive to determining that one or more HRV values of patient 102 are less than a HRV threshold during detection of sleep apnea episodes based on heart rate, processing circuitry 412 may determine that one or more verification conditions have been satisfied and detect sleep apnea episodes based on impedance. In yet another example, responsive to determining that a threshold number (e.g., 5, 10, etc.) and/or a threshold rate (e.g., 1 sleep apnea episode per 2 minutes) of sleep apnea episodes occurred during detection of sleep apnea episodes based on heart rate, processing circuitry 412 may determine that one or more verification conditions have been satisfied and detect sleep apnea episodes based on measured impedance. In yet another example, responsive to receipt of an input indicating that patient 102 has a prescription associated with a low HRV (e.g., beta-adrenoceptor blocking agents), processing circuitry 412 may determine that one or more verification conditions have been satisfied and detect sleep apnea episodes based on measured impedance.

In some examples, responsive to determining that the sleep apnea risk score value of patient 102 satisfy a sleep apnea risk threshold, processing circuitry 412 may detect sleep apnea episodes based on impedance. For example, processing circuitry 412 may determine patient 102 has a sleep apnea risk score of a “high-risk” of suffering a sleep apnea episode. Response to determining patient has a “high-risk” sleep apnea risk score, processing circuitry 412 may detect sleep apnea episodes based on impedance.

In some examples, responsive to determining that one or more verification conditions have been satisfied, processing circuitry 412 may control sensing circuitry 418 to sense one or more parameters, such as impedance, at a higher resolution and/or for a longer duration of time.

In some examples, responsive to determining that the sleep apnea risk score value of patient 102 satisfy a sleep apnea risk threshold, processing circuitry 412 may control sensing circuitry 418 to sense one or more parameters, such as impedance at a higher resolution and/or for a longer duration of time. In some examples, processing circuitry 412 may control sensing circuitry 418 to sense one or more parameters, such as impedance, at particular resolution and/or a particular duration of time based on a value of the sleep apnea risk score.

In some examples, processing circuitry 412 adjusting a particular resolution and/or a particular duration in which sensing circuitry 418 senses one or more parameters based on a particular sleep apnea risk score or determining that one or more verification conditions have been satisfied provides a personalized and individually optimized sleep apnea detection for patient 102.

In some examples, processing circuitry 412 may be configured to discard indications of sleep apnea episodes determined to be false-positives. For instance, processing circuitry 412 may determine the sleep apnea episodes occurring during a first period (and detected based on heart rate) to be false-positives if processing circuitry 412 does not detect (based on measured impedances) one or more sleep apnea episodes occurring during a second period (e.g., a period of time subsequent to the first period). In other words, processing circuitry 412 may be configured to discard from memory 416 of medical device 406 indications of the one or more sleep apnea episodes of patient 102 occurring during the first period in response to (1) detecting one or more sleep apnea episodes occurring during a first period and (2) not detecting one or more sleep apnea episodes of patient 102 occurring during a second period.

In some examples, processing circuitry 412 may be configured to generate an indication that patient 102 has experienced limb movement in response to determining one or more sleep apnea episodes to be false-positives. This is because limb movements may affect heart rate parameters (potentially causing processing circuitry 412 to detect a sleep apnea episode based on heart rate) but not impedance measurements. While a sleep apnea episode detected based on heart rate may be a false-positive because of limb movement, an indication that patient 102 is experiencing limb movement may be helpful to patient 102, a clinician, etc. Accordingly, processing circuitry 412 may be configured to generate an indication that patient 102 has experienced limb movement in response to (1) detecting one or more sleep apnea episodes of patient 102 occurring during a first period and (2) not detecting one or more sleep apnea episodes of patient 102 occurring during a second period.

In any case, after determining whether one or more sleep apnea episodes occurred based on measured impedances, processing circuitry 412 may be configured to stop detection of sleep apnea episodes based on measured impedances (e.g., to conserve power). For example, processing circuitry 412 may be configured to stop detection of sleep apnea episodes based on measured impedances in response to the second period lasting (e.g., continuing) for a predetermined duration. An example predetermined duration is 2 minutes. Other predetermined durations, such as 1 minute, 3 minutes, etc., are contemplated by this disclosure.

In some examples, processing circuitry 412 may be configured to stop detection of sleep apnea episodes based on measured impedances in response to the satisfaction of a condition. For example, processing circuitry 412 may be configured to stop detection of sleep apnea episodes based on measured impedances in response to processing circuitry 412 not detecting an AF episode based on the cardiac signal during the second period. This may be particularly useful in examples where processing circuitry 412 initiated detection of sleep apnea episodes based on measured impedances in response to processing circuitry 412 detecting one or more AF episodes during the first period (e.g., when processing circuitry 412 is sensing cardiac signals and detecting one or more sleep apnea episodes based on heart rate parameters).

In some examples, processing circuitry 412 may be configured to extend detection of sleep apnea episodes based on measured impedances in response to the satisfaction of a condition. For example, if processing circuitry 412 detects one or more sleep apnea episodes based on measured impedances during the first 2 minutes of the second period, processing circuitry 412 may extend detection of sleep apnea episodes based on measured impedances by 10 minutes, which may increase the number of detected sleep apnea episodes. On the other hand, if processing circuitry 412 does not detect, based on measured impedances, any sleep apnea episodes that occurred during the first 2 minutes of the second period (where in this example 2 minutes is the predetermined duration), processing circuitry 412 may stop detection of sleep apnea episodes based on measured impedances.

In some examples, processing circuitry 412 may be configured to detect sleep apnea episodes based on measured impedance periodically and for a predetermined duration. For example, if patient 102 is known to take any drugs that can lead to low HRV (e.g., as indicated by an input to medical device 406 indicating that patient 102 has a prescription associated with a low HRV), then processing circuitry 412 may detect sleep apnea episodes based on measured impedance for 3 minutes every 30 minutes when the patient is sleeping (or expected to be sleeping, such as at nighttime).

Processing circuitry 412 may be configured to generate an indication that one or more sleep apnea episodes have been detected based on heart rate and/or impedance. For example, responsive to detecting, based on heart rate, sleep apnea episodes that occurred during the first period and detecting, based on measured impedance, sleep apnea episodes that occurred during the second period, processing circuitry 412 may generate an indication that patient 102 has experienced one or more sleep apnea episodes during the first period and the second period. In another example, responsive to not detecting, based on heart rate, any sleep apnea episodes that occurred during the first period and detecting, based on measured impedance, sleep apnea episodes that occurred during the second period, processing circuitry 412 may generate an indication that patient 102 has at least experienced one or more sleep apnea episodes during the second period. In yet another example, responsive to detecting, based on heart rate, sleep apnea episodes that occurred during the first period and not detecting, based on measured impedance, any sleep apnea episodes that occurred during the second period, processing circuitry 412 may determine that the sleep apnea episodes detected during the first episode are false-positives and treat them accordingly (e.g., generate an indication that the sleep apnea episodes detected during the first period are likely to be the false-positives, discard the indications of the sleep apnea episodes during the first period, determine that the one or more sleep apnea episodes are limb movements and generate a corresponding indication, etc.).

It should be understood that any of the settings or thresholds described herein relating to the when and how processing circuitry 412 detects sleep apnea episodes based on measured impedances may be programmable and tuned individually for each patient (e.g., by the patient's clinician). Further, while this disclosure describes various examples in which processing circuitry 412 is configured to selectively detect sleep apnea episodes based on measured impedances to potentially reduce consumption of limited power and other resources of medical device 406, non-selective detection of sleep apnea episodes based on measured impedances for primary detection of sleep apnea episodes may be desirable in some circumstances. For example, if a clinician wants to know how many true sleep apnea episodes patient 102 is having, then the clinician can program medical device 406 to detect sleep apnea episodes based on measured impedance to detect the total number of sleep apnea events that patient 102 has for an entire night.

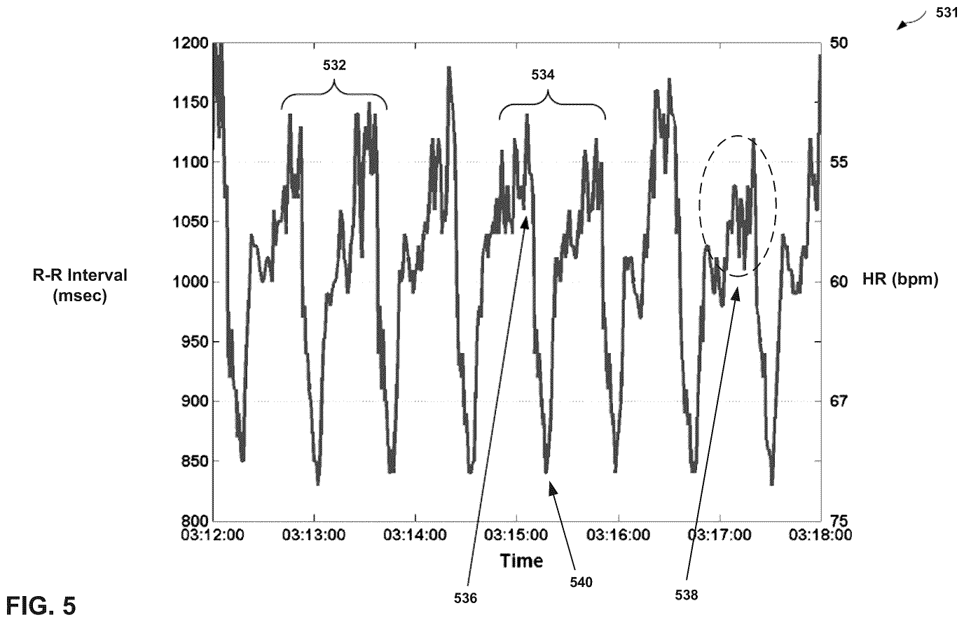

FIG. 5 is a graph 531 that illustrates sleep disordered heart rate (SDHR) information for patient. In FIG. 5, the R-R interval corresponds to the interval between two successive R-waves. In the example of graph 531, the SDHR of patient exhibits, at various times, characteristics of different physiological conditions. For instance, processing circuitry may analyze trends shown by clusters of maxima and minima (e.g., “peak” and “valley”) points of graph 531 to determine whether patient exhibits symptoms of a sleep apnea episode. For example, the processing circuitry of medical device 106 may use heart rate to the SDHR information in accordance with techniques of this disclosure. Some peak-and-valley clusters that the processing circuitry may analyze to detect possible sleep apnea episodes with respect to a patient are identified in FIG. 5.

For example, even during normal breathing, the heart rate information of graph 531 illustrates a type of cyclical variable heart rate (CVHR) referred to as respiratory sinus arrhythmia (RSA). During RSA, the heart rate of the patient increases with an inspiration (or inhalation) and the heart rate of patient decreases with expiration (or exhalation). RSA is associated with a single breath cycle, e.g., a single heart rate increase with inspiration and a single heart rate decrease with expiration. RSA is illustrated in the graph of FIG. 5 by higher frequency and lower amplitude variations 538.

The lower frequency and higher amplitude variations of the graph of FIG. 5, on the other hand, illustrate a form of CVHR, referred to herein as SDHR, associated with sleep apnea. For example, each of areas 532 and 534 illustrates one apnea arousal cycle, and the corresponding decrease and increase in heart rate. In most patients, a decrease of heart rate is observed during each apnea episode followed by an increase of heart rate near the end of the apnea episode. The heart rate further increases during ensuing hyperventilation. In the example of the individual sleep apnea episode associated with area 534 of graph 531, the heart rate increase due to autonomic arousal is indicated by crest 536. For instance, crest 536 may indicate a decrease that heart 104 experiences as the autonomic nervous system of patient attempts to compensate for the increased oxygen saturation during recovery from a sleep apnea episode. As such, troughs between such crests may be indicative of a period of overexertion of heart 104 which results from compensatory mechanisms that the autonomic nervous system of patient implements to counter the decreased oxygen saturation caused by the sleep apnea episode. As shown in FIG. 5, near the end of the sleep apnea episode (e.g., after a valley 540), the heart rate may increase.

Again, processing circuitry may collect and analyze physiological parameter value(s), determined in any way, to identify sleep apnea episodes based on the characteristics illustrated by the graph 531 of FIG. 5.

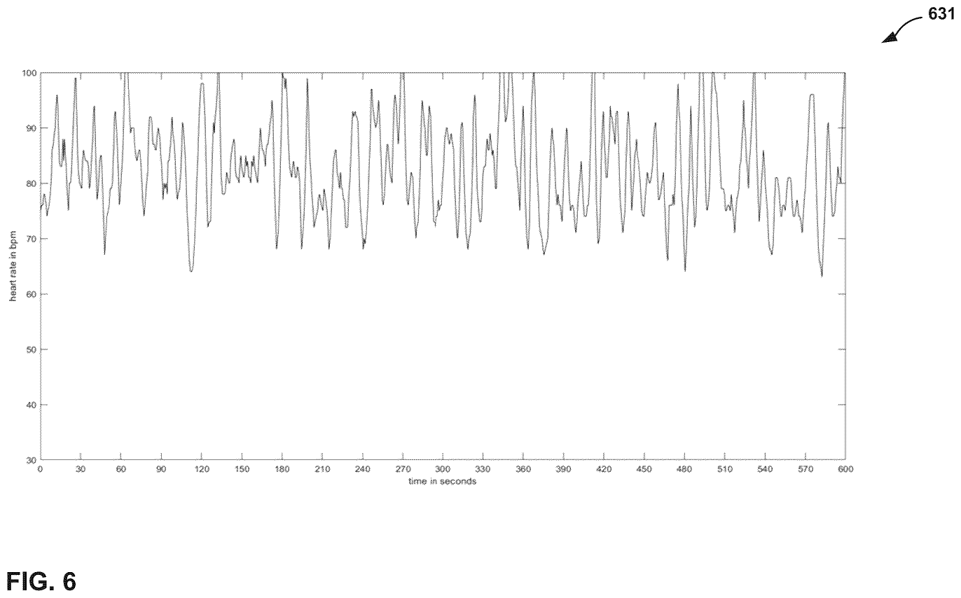

FIG. 6 is a graph 631 that illustrates cardiac electrogram signals indicative of AF. When a patient is in AF, the patient's heart rate may be irregular, which may cause a medical device system that detects sleep apnea episodes based on heart rate to fail to detect true sleep apnea episodes. For example, a medical device system that detects sleep apnea episodes based on heart rate may miss true sleep apnea episodes in a patient during AF or may falsely detect heart rate changes caused due to AF as sleep apnea episodes.

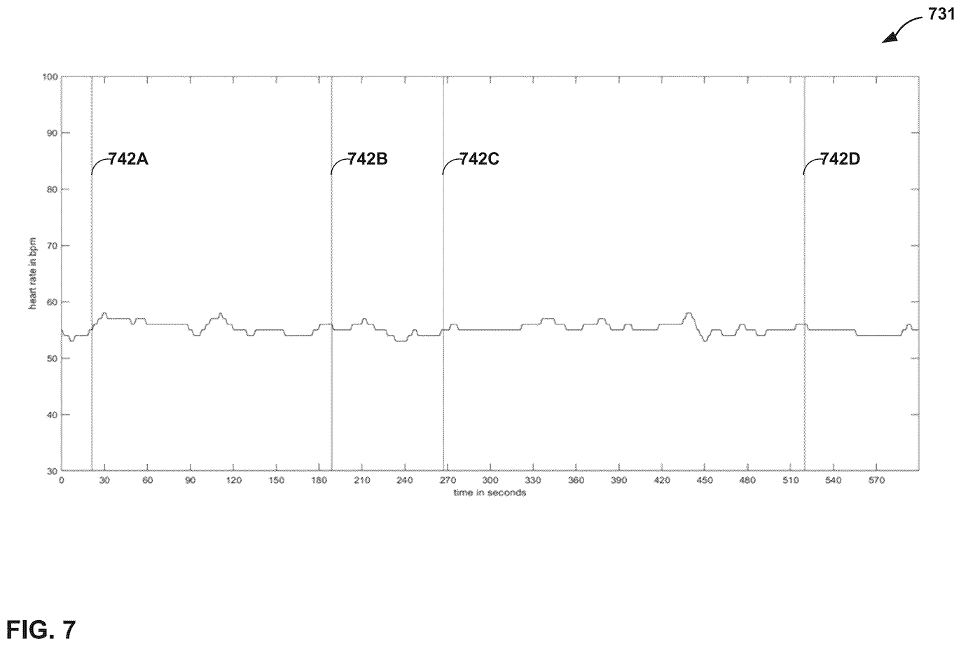

FIG. 7 is a graph that illustrates cardiac electrogram signals indicative of low heart variability. When a patient is taking certain medications such as high doses of beta blockers, the patient's heart rate may not change during sleep apnea episodes. Consequently, a medical device system that detects sleep apnea episodes based on heart rate may not be able to detect heart rate patterns that are expected during a sleep apnea episode and fail to detect true sleep apnea episodes. However, a medical device system that is configured to also detect sleep apnea episodes based on measured impedances (e.g., medical device system 100) may be able to detect sleep apnea episodes 742A-742D in the cardiac electrogram signals even when the patient has low heart variability because the measured impedances are not affected by medications that change heart rate.

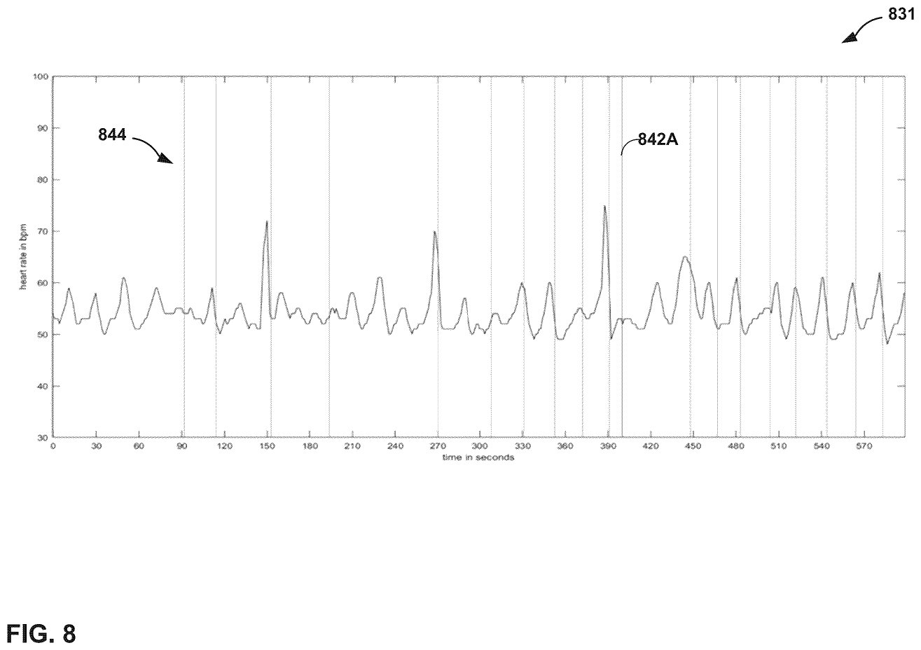

FIG. 8 is a graph that illustrates cardiac electrogram signals that may be analyzed for limb movement in accordance with techniques of this disclosure. During periodic limb movements, a patient's heart rate patterns may have similar characteristics to the heart rate patterns noticed during sleep apnea episodes. Consequently, a medical device system that detects sleep apnea episodes based on heart rate may incorrectly detect these events as sleep apnea episodes, leading to false positives. However, as described above, a medical device system that is configured to also detect sleep apnea episodes based on measured impedances (e.g., medical device system 100) may be able to confirm whether a sleep apnea episode detected based on heart rate is true or not. For example, as shown in FIG. 8, a medical device system that detects sleep apnea episodes based on measured impedances may determine that episode 842A (illustrated by a blue line) is a true sleep apnea episode and determine that episodes 844 (illustrated by an orange line) are false sleep apnea episodes. Further, the medical device system that detects sleep apnea episodes based on measured impedances may determine that episodes 844 are due to limb movements.

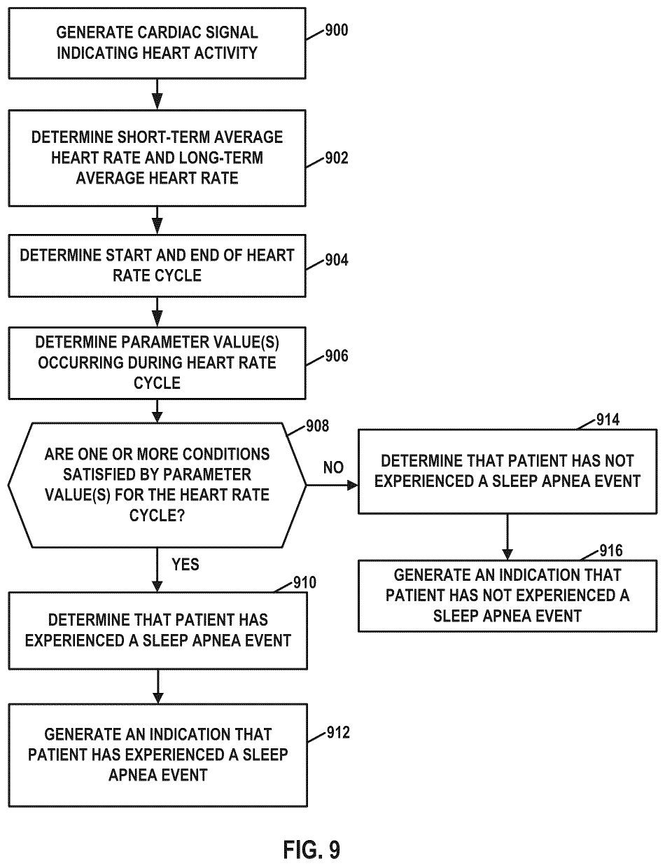

FIG. 9 is a flowchart illustrating a method by which a medical device system may use heart rate to detect a sleep apnea episode. Although described as being performed by medical device 406, one or more of the various example techniques described with reference to FIG. 9 may be performed by any one or more devices and/or systems including processing circuitry in accordance with techniques of this disclosure.

Medical device 406 may generate, by processing circuitry 412, a cardiac signal indicating activity of a heart of patient 102 in response to signals sensed by sensing circuitry 418 of medical device 406 (900). Medical device 406 may include one or more sensors 422, electrodes 430, etc., configured to sense signals produced by heart activity. Sensing circuitry 418 may deliver (e.g., send, transmit, etc.) detected signals to processing circuitry 412. Processing circuitry 412 may then generate a cardiac signal indicating the activity of a heart of patient 102. In some examples, the cardiac signal may be associated with characteristics of the heart activity of patient 102. For example, the cardiac signal may be associated with the waves, intervals, durations, and rhythm of the heart activity of patient 102.

Processing circuitry 412 may determine a short-term average heart rate and a long-term average heart rate upon which the start and end of a heart rate cycle may be based (902). The short-term average of the heart rate of patient 102 may be based on a first number of heartbeats, and the long-term average of the heart rate of patient 102 may be based on a second number of heartbeats, wherein the first number is less than the second number. For example, the first number of heartbeats may be equal to 3 heartbeats and the second number of heartbeats may be equal to 120 heartbeats. In such an example, the short-term average of the heart rate of patient 102 may be based on 3 heartbeats, and the long-term average of the heart rate of patient 102 may be based on 120 heartbeats. In other examples, the first number of heartbeats may be equal to a value other than 3 (e.g., 4, 5, etc.) and/or the second number of heartbeats may be equal to a value other than 120 (e.g., 119, 130, etc.).

Processing circuitry 412 may calculate the short-term average of the heart rate and the long-term average of the heart rate in various ways. For example, the short-term average of the heart rate and the long-term average of the heart rate may be the median of the first number of heartbeats and the second number of heartbeats, respectively (e.g., the median of 3 heartbeats and the median of 120 heartbeats, respectively). Alternatively, the short-term average of the heart rate and the long-term average of the heart rate may be the mean of the first number of heartbeats and the second number of heartbeats, respectively (e.g., the mean of 3 heartbeats and the mean of 120 heartbeats, respectively). Alternatively, the short-term average of the heart rate and the long-term average of the heart rate may be the mode of the first number of heartbeats and the second number of heartbeats (e.g., the mode of 3 heartbeats and the mode of 120 heartbeats, respectively). It should be understood that other methods for calculating the short-term average of the heart rate and the long-term average of the heart rate may be appropriate depending upon the circumstances.

Processing circuitry 412 may further determine the start and end of a heart rate cycle (904). The heart rate cycle defines a period of time of the patient 102's heart activity that is being processed by processing circuitry 412 to detect the occurrence of a sleep apnea episode. The start of the heart rate cycle may be based on a first time the short-term average of the heart rate of patient 102 changes from being less than the long-term average of the heart rate of patient 102 to being greater than the long-term average of the heart rate of patient 102. For example, if the long-term average of the heart rate of patient 102 is a constant 65 beats per minute (BPM), then the start of the heart rate cycle may be determined based on the first time that the short-term average of the heart rate of patient 102 exceeds 65 BPM. Similarly, the end of the heart rate cycle may be based on the second time the short-term average of the heart rate of patient 102 changes from being less than long-term average of the heart rate of patient 102 to being greater than the long-term average of the heart rate of patient 102. For example, if the long-term average of the heart rate of patient 102 is a constant 65 BPM, then the end of the heart rate cycle may be determined based on the second time that the short-term average of the heart rate of patient 102 exceeds 65 BPM.

Processing circuitry 412 may further determine corresponding parameter values for one or more heart rate parameters of patient 102 based on heart rate (and associated activity) occurring during the heart rate cycle (906). Determining whether patient 102 has experienced a sleep apnea episode during the first period may be based on the parameter values for the one or more heart rate parameters.

The parameter values determined by processing circuitry 412 may include a peak-to-valley time interval. The peak-to-valley time interval may be the time interval between a maximum short-term average of the heart rate during the heart rate cycle and a minimum short-term average of the heart rate during the heart rate cycle. For example, if a maximum short-term average of the heart rate value of 75 BPM occurs 30 seconds from a reference point (e.g., the beginning of a recording of the heart rate cycle, the beginning of the heart rate cycle, etc.) and during the heart rate cycle and the minimum short-term heart rate average value of 60 BPM occurs 45 seconds from the reference point and during the heart rate cycle, then the peak-to-valley time interval is equal to 15 seconds.

The parameter values determined by processing circuitry 412 may further include an activity count. The activity count for the heart rate cycle may indicate a number of time intervals during the heart rate cycle in which an amount of movement of the patient 102 is greater than a minimum movement threshold. Amount of movement of patient 102 may be determined using one or more sensors 422 (e.g., accelerometer 416). For example, accelerometer 416 may measure the acceleration of patient's body, and if the acceleration of patient's body exceeds an acceleration threshold corresponding to a minimum movement threshold, the activity count for the heart rate cycle may be incremented by one. As such, if, in a single heart rate cycle, the acceleration of patient's body exceeds the acceleration threshold 8 times, the minimum movement threshold may also be exceeded 8 times so that the activity count for patient 102 is 8.

The parameter values determined by processing circuitry 412 may further include a peak-to-valley heart rate variation value. The peak-to-valley heart rate variation value for the heart rate cycle may be the difference between a maximum short-term average of the heart rate during the heart rate cycle and a minimum short-term average of the heart rate during the heart rate cycle. For example, if the maximum short-term average of the heart rate during the heart rate cycle is 75 BPM and the minimum short-term average of the heart rate during the heart rate cycle is 60 BPM, then the peak-to-valley heart rate variation value is 15 BPM for this heart rate cycle.

The parameter values determined by processing circuitry 412 may further include a cycle length, where the cycle length for the heart rate cycle indicates a length of the heart rate cycle. For example, if the start of the heart rate cycle is at first time of 0 seconds (e.g., based on the first time that the short-term average of the heart rate of patient 102 exceeds the long-term average of the heart rate of patient 102), and the end of the heart rate cycle is at second time of 70 seconds (e.g., based on the second time that the short-term average of the heart rate of patient 102 exceeds the long-term average of the heart rate of patient 102), the cycle length is 70 seconds.

In some examples, processing circuitry 412 may derive a parameter value from other parameter values. For example, processing circuitry 412 may use a series of heart rate values to determine the peak-to-valley time interval, which in turn may be used to detect a sleep apnea episode. For example, if the short-term average of the heart rate of patient 102 fluctuates from 70 BPM to 60 BPM, 60 BPM to 85 BPM, 85 BPM to 65 BPM, and so on during the heart cycle, heart rate values may include 10 BPM (70 BPM less 60 BPM), 25 BPM (85 BPM less 60 BPM), 5 BPM (70 BPM less 65 BPM), and so on. In this example, the peak-to-valley variation value is 25 BPM because 85 BPM is the maximum short-term heart rate average value during the heart rate cycle, and 60 BPM is the minimum short-term average value during the heart rate cycle.

Processing circuitry 412 may then use the respective times that the maximum short-term average of the heart rate value of 85 BPM and the minimum short-term average of the heart rate value of 60 BPM occurred to determine the peak-to-valley time interval. In some examples, the peak-to-valley time interval may equal the time the valley occurred subtracted by the time the peak occurred. For example, if the maximum short-term average of the heart rate value of 85 BPM occurred 30 seconds from the start of the heart rate cycle and the minimum short-term average of the heart rate value of 60 BPM occurred 60 seconds from the start of the heart rate cycle, then the peak-to-valley time interval is equal to 30 seconds.

Processing circuitry 412 may further determine whether one or more conditions of a plurality of conditions are satisfied by one or more parameter values for patient 102 for the heart rate cycle. The plurality of conditions may include, but is not limited to, a peak-to-valley time interval condition, an activity count condition, a peak-to-valley heart rate variation condition, a cycle length condition, etc.

The plurality of conditions may include a peak-to-valley time interval condition. The peak-to-valley time interval condition may be a condition that the peak-to-valley time interval is greater than a lower peak-to-valley time threshold and less than an upper peak-to-valley time threshold. For example, if the lower peak-to-valley time threshold is equal to 5 seconds and the upper peak-to-valley time threshold is equal to 30 seconds, then processing circuitry 412 may determine that the peak-to-valley time interval condition is satisfied if the peak-to-valley time interval is greater than 5 seconds and less than 30 seconds. As such, a peak-to-valley time interval of 10 seconds, for example, would satisfy the peak-to-valley time interval condition. Alternatively, processing circuitry 412 may determine that the peak-to-valley time interval condition is not satisfied if the peak-to-valley time interval is not greater than 5 seconds and less than 30 seconds. As such, a peak-to-valley time interval of 3 seconds, for example, would not satisfy the peak-to-valley time interval condition.

Additionally or alternatively, the plurality of conditions may include an activity count condition. The activity count condition may be a condition that an activity count for the heart rate cycle is less than an activity count threshold. For example, if the activity count threshold is equal to 8, then processing circuitry 412 may determine that the activity count condition is satisfied if the activity count is less than 8. As such, an activity count of 5, for example, would satisfy the activity count condition. Alternatively, processing circuitry 412 may determine that the activity count condition is not satisfied if the activity count is not less than 8. As such, an activity count of 10, for example, would not satisfy the activity count condition.

Additionally or alternatively, the plurality of conditions may include a peak-to-valley HRV condition. The peak-to-valley HRV condition may be a condition that the peak-to-valley HRV value for the heart rate cycle is greater than a lower peak-to-valley heart rate variation threshold and less than an upper peak-to-valley heart rate variation threshold. For example, if the lower peak-to-valley heart rate variation threshold is equal to 6 BPM and the upper peak-to-valley heart rate variation threshold is equal to 50 BPM, then processing circuitry 412 may determine that the peak-to-valley HRV condition is satisfied if the peak-to-valley HRV value is greater than 6 and less than 50. As such, a peak-to-valley HRV value of 25, for example, would satisfy the peak-to-valley HRV condition. Alternatively, processing circuitry 412 may determine that the peak-to-valley HRV condition is not satisfied if the peak-to-valley HRV value is not less than 50. As such, a peak-to-valley HRV value of 60, for example, would not satisfy the peak-to-valley HRV condition.

Additionally or alternatively, the plurality of conditions may include a cycle length condition. The cycle length is the length of the heart rate cycle (e.g., the length of the period of time of patient 102's heart activity being processed by processing circuitry 412 for detecting a sleep apnea episode). The cycle length condition may be a condition that the cycle length for the heart rate cycle is greater than a lower cycle length threshold and less than an upper cycle length threshold. For example, if the lower cycle length threshold is equal to 25 seconds and the upper cycle length threshold is equal to 100 seconds, then processing circuitry 412 may determine that the cycle length condition is satisfied if the cycle length is greater than 25 seconds and less than 100 seconds. As such, a cycle length of 50 seconds, for example, would satisfy the cycle length condition. Alternatively, processing circuitry 412 determine that the cycle length condition is not satisfied if the cycle length is not greater than 25 seconds and less than 100 seconds. As such, a cycle length of 125 seconds, for example, would not satisfy the cycle length condition.

In some examples, the plurality of conditions may include the peak-to-valley time interval condition, the activity count condition, the peak-to-valley heart rate variation condition, and the cycle length condition. In such examples, determining that the patient has experienced the sleep apnea episode may be based at least in part on each of the conditions of the plurality of conditions being satisfied for the heart rate cycle.

In some examples, processing circuitry 412 may further determine whether a patient 102 has experienced a sleep apnea episode based on whether one or more conditions of the plurality of conditions are satisfied (908). For example, if each of the one or more conditions is satisfied (e.g., peak-to-valley time interval condition is satisfied) (“YES” branch of 908), processing circuitry 412 may determine that patient 102 has experienced a sleep apnea episode or that the probability that patient 102 has experienced a sleep apnea episode is not low (910). Processing circuitry 412 may then cause the generation of an indication that patient 102 has experienced a sleep apnea episode (912). For example, processing circuitry 412 may cause medical device 406 and/or external device 408 to generate an indication that patient 102 has experienced a sleep apnea episode.

Alternatively, if one or more of the conditions have not been satisfied (“NO” branch of 908), processing circuitry 412 may determine that patient 102 has not experienced a sleep apnea episode or that the probability that patient 102 has experienced a sleep apnea episode is low (914). Processing circuitry 412 may then cause the generation of an indication that patient 102 has not experienced a sleep apnea episode (916). For example, processing circuitry 412 may cause medical device 406 and/or external device 408 to generate an indication that patient 102 has not experienced a sleep apnea episode. Alternatively, in some examples, processing circuitry 412 does not generate an indication that patient 102 has experienced a sleep apnea episode if patient 102 has not experienced a sleep apnea episode.

Processing circuitry 412 may generate an indication that patient 102 has experienced a sleep apnea episode. For example, processing circuitry 412 may cause the medical device or an external device to output an indication that patient 102 has experienced a sleep apnea episode. The external device may include one or more cellular phones, a ‘smartphone,’ a satellite phone, a notebook computer, a tablet computer, a wearable device, a computer workstation, a personal digital assistant, a handheld computing device, a virtual reality headset, or any other device that may output an indication that patient 102 has experienced a sleep apnea episode. External device may output the indication automatically (e.g., at a pre-determined time of day) and/or in response to input from patient 102 as part of a report or history of patient and in the form of an audible notification, a visual notification, a tactile notification (e.g., a vibration or vibrational pattern), a text prompt, a button prompt, and/or any other notification that may indicate to patient 102 that patient 102 has experienced a sleep apnea episode.

Although not illustrated in FIG. 9, processing circuitry 412 may determine a confidence value, such as a probability, that patient 102 has experienced a sleep apnea episode based on the number of conditions of the plurality of conditions that are satisfied. In some instances, the confidence value may be positively correlated with the number of conditions that are satisfied. For example, if only one condition is satisfied, processing circuitry 412 may determine that the confidence value that patient 102 has experienced a sleep apnea episode is low. In another example, if two conditions are satisfied, processing circuitry 412 may determine that the confidence value that patient 102 has experienced a sleep apnea episode is moderate. In yet another example, if three or more conditions are satisfied, processing circuitry 412 may determine that the confidence value that patient 102 has experienced a sleep apnea episode is high.

Although not illustrated in FIG. 9, processing circuitry 412 may determine that patient 102 has experienced a sleep apnea episode based on one or more conditions being satisfied by at least a non-zero minimum number of additional heart rate cycles other than the current heart rate cycle that occur within a predetermined temporal distance (e.g., amount of time) of the current heart rate cycle. For example, if the minimum number of additional heart rate cycles is equal to 2 and the predetermined temporal distance is equal to 240 seconds, then processing circuitry 412 may determine that patient 102 has experienced a sleep apnea episode based on one or more conditions (e.g., the peak-to-valley time interval condition, the activity count condition, the peak-to-valley heart rate variation condition, the cycle length condition, etc.) being satisfied by 2 additional heart rate cycles other than the current heart rate cycle that occur within 240 seconds of the current heart rate cycle. As such, processing circuitry 412 may determine that patient 102 has experienced a sleep apnea episode based on the activity count condition being satisfied by a heart rate cycle with a temporal distance of 220 seconds of the current heart rate cycle and the peak-to-valley heart rate variation condition being satisfied by a heart rate cycle with a temporal distance of 75 seconds of the current heart rate cycle. Alternatively, processing circuitry 412 may determine that patient 102 has not experienced a sleep apnea episode based on the activity count condition being satisfied by a heart rate cycle with a temporal distance of 300 seconds of the current heart rate cycle and the peak-to-valley heart rate variation condition being satisfied by a heart rate cycle with a temporal distance of 75 seconds of the current heart rate cycle because the heart rate cycle with a temporal distance of 300 seconds did not occur within the predetermined temporal distance of 240 seconds. Additionally or alternatively, in the same example, processing circuitry 412 may determine that patient 102 has not experienced a sleep apnea episode because one or more conditions were not satisfied by 2 or more additional heart rate cycles (e.g., one condition was only satisfied by 1 additional heart rate cycle because only the activity count condition was satisfied by only 1 heart rate cycle with a temporal distance of 75 seconds of the current heart rate cycle).

The reference point for determining the temporal distance may vary. For example, the reference point for determining the temporal distance may be the start of a heart rate cycle, the end of the heart rate cycle, and any point in between the start and end of the heart rate cycle. In some examples, the direction of the temporal distance may vary. That is, the direction of the temporal distance may be retrospective so that heart activity that already occurred may be evaluated, prospective so that heart activity that will occur may be evaluated, or a combination of both.

For example, if the reference point for determining the temporal distance is the start of the heart rate cycle and the predetermined temporal distance is 240 seconds, then processing circuitry 412 may determine that patient 102 has experienced a sleep apnea episode based on heart activity that occurred within any heart rate cycle before the start of the current heart rate cycle with a temporal distance of 240 seconds or less. In another example, processing circuitry 412 may determine that patient 102 has experienced a sleep apnea episode based on heart activity that occurred within any heart rate cycle after the start of the current heart rate cycle with a temporal distance of 240 seconds or less. In yet another example, processing circuitry 412 may determine that patient 102 has experienced a sleep apnea episode based on heart activity occurring within any heart rate cycle before the start of the current heart rate cycle and after the start of the current heart cycle, as long as the heart activity satisfying the one or more conditions occur within a temporal distance of 240 seconds or less.

In another example, if the reference point for determining the temporal distance is the end of the heart rate cycle and the predetermined temporal distance is 240 seconds, then processing circuitry 412 may determine that patient 102 has experienced a sleep apnea episode based on heart activity that occurred within any heart rate cycle before the end of the current heart rate cycle with a temporal distance of 240 seconds or less. Alternatively, processing circuitry 412 may determine that patient 102 has experienced a sleep apnea episode based on heart activity that occurred within any heart rate cycle after the end of the current heart rate cycle with a temporal distance of 240 seconds or less. Alternatively, processing circuitry 412 may determine that patient 102 has experienced a sleep apnea episode based on heart activity that occurred within any heart rate cycle before the end of the current heart rate cycle with a temporal distance of 120 seconds or less and within any heart rate cycle after the end of the current heart rate cycle with a temporal distance of 120 seconds or less.

In another example, if the reference point for determining the temporal distance is the midpoint of the heart rate cycle and the predetermined temporal distance is 240 seconds, then processing circuitry 412 may determine that patient 102 has experienced a sleep apnea episode based on heart activity that occurred within any heart rate cycle before the midpoint of the current heart rate cycle with a temporal distance of 240 seconds or less. Alternatively, processing circuitry 412 may determine that patient 102 has experienced a sleep apnea episode based on heart activity that occurred within any heart rate cycle after the midpoint of the current heart rate cycle with a temporal distance of 240 seconds or less. Alternatively, processing circuitry 412 may determine that patient 102 has experienced a sleep apnea episode based on heart activity that occurred within any heart rate cycle before the midpoint of the current heart rate cycle with a temporal distance of 120 seconds or less and within any heart rate cycle after the midpoint of the current heart rate cycle with a temporal distance of 120 seconds or less.

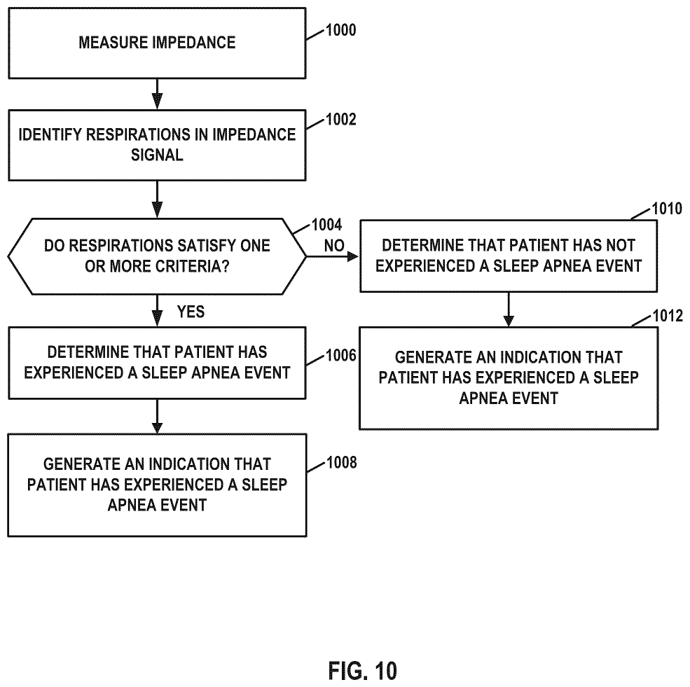

FIG. 10 is a flowchart illustrating a method by which a medical device system may use impedance to detect a sleep apnea episode. Although described as being performed by medical device 406, one or more of the various example techniques described with reference to FIG. 10 may be performed by any one or more devices and/or systems including processing circuitry in accordance with techniques of this disclosure.

Processing circuitry 412 may control sensing circuitry 418 to measure an impedance signal (1000). Processing circuitry 412 may use impedance to detect one or more sleep apnea episodes based on the impedance signal. For example, processing circuitry 412 may detect respirations based on the measured impedances of patient 102 (1002). Processing circuitry 412 may analyze this pattern of inhalation and exhalation and identify anomalies indicative of an absence of respiration when patient 102 is sleeping (i.e., a sleep apnea episode). An example anomaly may include an abnormally long interval between inhalations and/or exhalations indicative of a skipped inhalation and/or exhalation.

Processing circuitry 412 may determine whether the detected respirations satisfy one or more criteria (e.g., whether an inhalation and/or exhalation has been skipped). Responsive to determining that the detected respirations satisfy one or more criteria (“YES” branch of 1004), processing circuitry 412 may determine that patient 102 has experienced a sleep apnea episode (1006). Processing circuitry 412 may then cause the generation of an indication that patient 102 has experienced a sleep apnea episode (1008). For example, processing circuitry 412 may cause medical device 406 and/or external device 408 to generate an indication that patient 102 has experienced a sleep apnea episode.

Alternatively, responsive to determining that the detected respirations do not satisfy one or more criteria (“NO” branch of 1004), processing circuitry 412 may determine that patient 102 has not experienced a sleep apnea episode (1010). Processing circuitry 412 may then cause the generation of an indication that patient 102 has not experienced a sleep apnea episode (1012). For example, processing circuitry 412 may cause medical device 406 and/or external device 408 to generate an indication that patient 102 has not experienced a sleep apnea episode. Alternatively, in some examples, processing circuitry 412 does not generate an indication that patient 102 has experienced a sleep apnea episode if patient 102 has not experienced a sleep apnea episode.

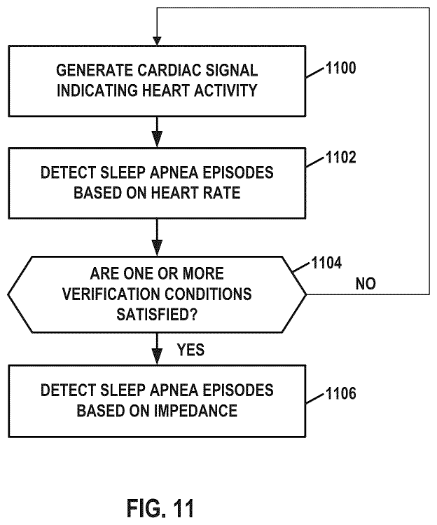

FIG. 11 is a flowchart illustrating a method by which a medical device system may use heart rate and impedance to detect a sleep apnea episode. Although described as being performed by medical device 406, one or more of the various example techniques described with reference to FIG. 10 may be performed by any one or more devices and/or systems including processing circuitry in accordance with techniques of this disclosure.

Medical device system 406 may generate, by processing circuitry 412, a cardiac signal indicating activity of a heart of patient 102 in response to signals sensed by sensing circuitry 418 of medical device 406 (1100). As described above (e.g., with respect to FIG. 9), medical device 406 may use heart rate to the cardiac signal during a first period of time (1102).

Processing circuitry 412 may determine whether one or more verification conditions are satisfied. Example verification conditions may include, but are not limited to, detection of one or more AF episodes during the first period, a threshold number (e.g., 5, 10, etc.) or a threshold rate (e.g., 1 sleep apnea episode per 2 minutes) of sleep apnea episodes occurring during the first period, receipt of an input indicating that patient 102 has a prescription associated with a low heart rate variability (e.g., beta-adrenoceptor blocking agents), one or more HRV values of patient 102 being less than a HRV threshold, and user input.

Responsive to determining that the one or more verification conditions are satisfied (“YES” branch of 1104), processing circuitry 412 may control sensing circuitry 418 to measure impedance during a second period of time. processing circuitry 412 may then use impedance to detect one or more sleep apnea episodes during the second period of time (1106).

Responsive to determining that the one or more verification conditions are not satisfied (“NO” branch of 1104), processing circuitry 412 may not detect sleep apnea episodes based on measured impedance (e.g., to conserve power). For example, processing circuitry 412 may continue generating a cardiac signal indicative of heart activity (1100) and detect sleep apnea episodes based on heart rate.

In some examples, processing circuitry 412 may discard indications of sleep apnea episodes determined to be false-positives. For instance, processing circuitry 412 may determine the sleep apnea episodes detected based on heart rate during a first period to be false-positives if processing circuitry 412 does not detect, based on measured impedances, one or more sleep apnea episodes of patient 102 occurring during a second period (e.g., a period of time subsequent to the first period). In some examples, processing circuitry 412 generate an indication that patient 102 has experienced limb movement in response to determining one or more sleep apnea episodes to be false-positives.

In any case, processing circuitry 412 may be configured to generate an indication that one or more sleep apnea episodes have been detected based on heart rate and/or impedance. For example, responsive to detecting, based on heart rate, sleep apnea episodes that occurred during the first period and detecting, based on measured impedance, sleep apnea episodes that occurred during the second period, processing circuitry 412 may generate an indication that patient 102 has experienced one or more sleep apnea episodes during the first period and the second period. In another example, responsive to not detecting, based on heart rate, any sleep apnea episodes that occurred during the first period and detecting, based on measured impedance, sleep apnea episodes that occurred during the second period, processing circuitry 412 may generate an indication that patient 102 has at least experienced one or more sleep apnea episodes during the second period. In yet another example, responsive to detecting, based on heart rate, sleep apnea episodes that occurred during the first period and not detecting, based on measured impedance, any sleep apnea episodes that occurred during the second period, processing circuitry 412 may determine that the sleep apnea episodes detected during the first episode are false-positives and treat them accordingly (e.g., generate an indication that the sleep apnea episodes detected during the first period are likely to be the false-positives, discard the indications of the sleep apnea episodes during the first period, determine that the one or more sleep apnea episodes are limb movements and generate a corresponding indication, etc.).

In some examples, 412 may detect sleep apnea episodes based on heart rate and/or impedance by implementing a machine learned model or other machine learned or artificial intelligence (AI) algorithm. Example machine learning techniques that may be employed to generate such algorithms can include various learning styles, such as supervised learning, unsupervised learning, and semi-supervised learning. Example types of algorithms include Bayesian algorithms, Clustering algorithms, decision-tree algorithms, regularization algorithms, regression algorithms, instance-based algorithms, artificial neural network algorithms, deep learning algorithms, dimensionality reduction algorithms and the like. Various examples of specific algorithms include Bayesian Linear Regression, Boosted Decision Tree Regression, and Neural Network Regression, Back Propagation Neural Networks, Convolution Neural Networks (CNN), Long Short Term Networks (LSTM), the Apriori algorithm, K-Means Clustering, k-Nearest Neighbour (kNN), Learning Vector Quantization (LVQ), Self-Organizing Map (SOM), Locally Weighted Learning (LWL), Ridge Regression, Least Absolute Shrinkage and Selection Operator (LASSO), Elastic Net, and Least-Angle Regression (LARS), Principal Component Analysis (PCA) and Principal Component Regression (PCR).

The input(s) to such an algorithm may include one or more vectors of data, such as cardiac EGM signal samples, values of the heart rate and/or HRV parameters described herein, or impedance signal samples. In some examples, the impedance-based algorithm may, in addition to the impedance signal, accept cardiac EGM or other metrics derived therefrom as inputs. An output of such an algorithm may be a value representing a likelihood, probability, and/or confidence of one or more classifications, such as “sleep apnea” or “not sleep apnea.” As one example, such an algorithm may include a CNN or other deep learning model trained with numerous training data samples labeled as “sleep apnea” or “not sleep apnea.”

As used herein, an implantable medical device (IMD) may include, be, or be part of a variety of devices or integrated systems, such as, but not limited to, implantable cardiac monitors (ICMs), implantable pacemakers, including those that deliver cardiac resynchronization therapy (CRT), implantable cardioverter defibrillators (ICDs), diagnostics device, cardiac device, etc. Various examples have been described that include detecting episodes of sleep apnea using cardiac cycle length metrics. In addition, pulmonary therapy may be provided to mitigate the severity of the sleep apnea episode or counter the effects of the sleep apnea episode. Any combination of detection and therapy for sleep apnea episodes is contemplated.

Various aspects of the techniques may be implemented within one or more processing circuitries, including one or more microprocessors, DSPs, ASICs, FPGAs, or any other equivalent integrated or discrete logic circuitry, as well as any combinations of such components, embodied in external devices, such as clinician or patient external devices, electrical stimulators, or other devices. The term “processing circuitry” may generally refer to any of the foregoing logic circuitry, alone or in combination with other logic circuitry or any other equivalent circuitry.

In one or more examples, the functions described in this disclosure may be implemented in hardware, software, firmware, or any combination thereof. If implemented in software, the functions may be stored on, as one or more instructions or code, a computer-readable medium and executed by a hardware-based processing unit. Computer-readable media may include computer-readable storage media forming a tangible, non-transitory medium. Instructions may be executed by one or more processing circuitries, such as one or more DSPs, ASICs, FPGAs, general purpose microprocessors, or other equivalent integrated or discrete logic circuitry. Accordingly, the term “processing circuitry,” as used herein may refer to one or more of any of the foregoing structure or any other structure suitable for implementation of the techniques described herein.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external device, a combination of an IMD and external device, an integrated circuit (IC) or a set of ICs, and/or discrete electrical circuitry, residing in an IMD and/or external device.

The following examples are a non-limiting list of clauses in accordance with one or more techniques of this disclosure.

Example 1. A medical device comprising: one or more electrodes; sensing circuitry configured to: sense a cardiac signal indicating activity of a heart of a patient; and measure impedances of the patient via the one or more electrodes; and processing circuitry configured to: determine heart rates of the patient based on the cardiac signal sensed during a first period; detect one or more sleep apnea episodes of the patient occurring during the first period based on the heart rates; determine whether one or more verification conditions are satisfied; and responsive to determining that the one or more verification conditions are satisfied: control the sensing circuitry to measure impedances of the patient during a second period subsequent to the first period; and detect one or more sleep apnea episodes of the patient occurring during the second period based on the measured impedances.

Example 2. The medical device of Example 1, wherein the processing circuitry is further configured to detect atrial fibrillation episodes based on the cardiac signal, and wherein the one or more verification conditions comprise detection of an atrial fibrillation episode during the first period.

Example 3. The medical device of Example 1 or 2, wherein the one or more verification conditions comprise detection of a threshold number or a threshold rate of sleep apnea episodes occurring during the first period.

Example 4. The medical device of Example 3, wherein the threshold number is 10.

Example 5. The medical device of Example 4, wherein the threshold rate is 1 sleep apnea episode per 2 minutes.

Example 6. The medical device of any of Examples 1 to 5, wherein the one or more verification conditions comprise receipt, by the processing circuitry, of an input indicating that the patient has a prescription associated with a low heart rate variability.

Example 7. The medical device of any of Examples 1 to 6, wherein the processing circuitry is configured to determine one or more heart rate parameters based on the determined heart rates of the patient, and wherein the processing circuitry is configured to detect the one or more sleep apnea episodes of the patient occurring during the first period based on the determined heart rates by detecting the one or more sleep apnea episodes of the patient occurring during the first period based on the one or more heart rate parameters.

Example 8. The medical device of any of Examples 1 to 7, wherein the processing circuitry is configured to detect respirations based on the measured impedances of the patient, and wherein the processing circuitry is configured to detect the one or more sleep apnea episodes of the patient occurring during the second period based on the measured impedances by detecting the one or more sleep apnea episodes of the patient occurring during the second period based on the respirations.

Example 9. The medical device of any of Examples 1 to 8, wherein the processing circuitry is further configured to determine a heart rate variability value based on the heart rates, wherein the one or more verification conditions comprise the heart rate variability value being less than the heart rate variability threshold.

Example 10. The medical device of any of Examples 1 to 9, wherein the processing circuitry is further configured to, responsive to (1) detecting the one or more sleep apnea episodes of the patient occurring during the first period and (2) not detecting the one or more sleep apnea episodes of the patient occurring during the second period, discard from memory circuitry of the medical device indications of the one or more sleep apnea episodes of the patient occurring during the first period.

Example 11. The method of any of Examples 1 to 10, wherein the processing circuitry is further configured to, responsive to (1) detecting the one or more sleep apnea episodes of the patient occurring during the first period and (2) not detecting the one or more sleep apnea episodes of the patient occurring during the second period, generate an indication that the patient has experienced limb movement during the first period.

Example 12. The medical device of any of Examples 1 to 11, wherein the processing circuitry is further configured to stop detection of sleep apnea episodes based on the measured impedances in response to the second period lasting for a predetermined duration.

Example 13. The medical device of Example 12, wherein the pre-determined duration is 2 minutes.

Example 14. The medical device of any of Examples 1 to 13, wherein the processing circuitry is further configured to, responsive to not detecting an atrial fibrillation episode based on the cardiac signal during the second period, stop detection of sleep apnea episodes based on the measured impedances.

Example 15. A method comprising: sensing, by sensing circuitry of a medical device, a cardiac signal indicating activity of a heart of a patient; determining, by processing circuitry of the medical device, heart rates of the patient based on the cardiac signal sensed during a first period; detecting, by the processing circuitry, one or more sleep apnea episodes of the patient occurring during the first period based on the heart rates; determining, by the processing circuitry, whether one or more verification conditions are satisfied; and responsive to determining that the one or more verification conditions are satisfied: controlling, by the processing circuitry, the sensing circuitry to measure impedances of the patient during a second period subsequent to the first period; and determining whether one or more sleep apnea episodes of the patient occurred during the second period based on the measured impedances.

Example 16. The method of Example 15, further comprising detecting, by the processing circuitry, atrial fibrillation episodes based on the cardiac signal, wherein the one or more verification conditions comprise detection of an atrial fibrillation episode during the first period.

Example 17. The method of Example 15 or 16, wherein the one or more verification conditions comprise detection of a threshold number or threshold rate of sleep apnea episodes occurring during the first period.

Example 18. The method of Example 17, wherein the threshold number is 10.

Example 19. The method of any of Example 17 or 18, wherein the threshold rate is 1 sleep apnea episode per 2 minutes.

Example 20. The method of any of Examples 15 to 19, wherein the one or more verification conditions comprise receipt, by the processing circuitry, of an input indicating that the patient has a prescription associated with a low heart rate variability.

Example 21. The method of any of Example 15 to 20, further comprising determining one or more heart rate parameters based on the heart rates of the patient, and wherein detecting the one or more sleep apnea episodes of the patient occurring during the first period based on the heart rates comprises detecting the one or more sleep apnea episodes of the patient occurring during the first period based on the one or more heart rate parameters.

Example 22. The method of any of Examples 15 to 21, further comprising detecting respirations based on the measured impedances of the patient, and wherein detecting the one or more sleep apnea episodes of the patient occurring during the second period based on the measured impedances comprises detecting the one or more sleep apnea episodes of the patient occurring during the second period based on the respirations.

Example 23. The method of any of Examples 15 to 22, further comprising: determining, by the processing circuitry, a heart rate variability value based on the heart rates; and comparing, by the processing circuitry, the heart rate variability value to a heart rate variability threshold, wherein the one or more verification conditions comprise the heart rate variability value being less than the heart rate variability threshold.

Example 24. The method of any of Examples 15 to 23, further comprising, responsive to (1) detecting the one or more sleep apnea episodes of the patient occurring during the first period and (2) not detecting the one or more sleep apnea episodes of the patient occurring during the second period, discarding, by the processing circuitry and from memory circuitry of the medical device, indications of the one or more sleep apnea episodes of the patient occurring during the first period.

Example 25. The method of any of Examples 15 to 24, further comprising, responsive to (1) detecting the one or more sleep apnea episodes of the patient occurring during the first period and (2) not detecting the one or more sleep apnea episodes of the patient occurring during the second period, generating, by the processing circuitry, an indication that the patient has experienced limb movement during the first period.

Example 26. The method of any of Examples 15 to 25, further comprising stopping, by the processing circuitry, detection of sleep apnea episodes based on the measured impedances in response to the second period lasting for a predetermined duration.

Example 27. The method of any of Examples 15 to 26, wherein the pre-determined duration is 2 minutes.

Example 28. The method of any of Examples 15 to 27, further comprising stopping, by the processing circuitry, detection of sleep apnea episodes based on the measured impedances in response to not detecting an atrial fibrillation episode based on the cardiac signal during the second period.

What is claimed is:

1. A medical device comprising:
one or more electrodes;
sensing circuitry configured to:
sense a cardiac signal indicating activity of a heart of a patient; and
measure impedances of the patient via the one or more electrodes; and
processing circuitry configured to:
determine heart rates of the patient based on the cardiac signal sensed during a first period;
detect one or more sleep apnea episodes of the patient occurring during the first period based on the heart rates;
determine whether one or more verification conditions are satisfied, wherein the one or more verification conditions comprise detection of a threshold number or a threshold rate of sleep apnea episodes occurring during the first period; and
responsive to determining that the one or more verification conditions are satisfied:
control the sensing circuitry to measure impedances of the patient during a second period subsequent to the first period; and
detect one or more sleep apnea episodes of the patient occurring during the second period based on the measured impedances.

2. The medical device of claim 1, wherein the processing circuitry is further configured to detect atrial fibrillation episodes based on the cardiac signal, and wherein the one or more verification conditions comprise detection of an atrial fibrillation episode during the first period.

3. The medical device of claim 1, wherein the threshold number is 10.

4. The medical device of claim 3, wherein the threshold rate is 1 sleep apnea episode per 2 minutes.

5. The medical device of claim 1, wherein the one or more verification conditions comprise receipt, by the processing circuitry, of an input indicating that the patient has a prescription associated with a low heart rate variability.

6. The medical device of claim 1, wherein the processing circuitry is configured to determine one or more heart rate parameters based on the determined heart rates of the patient, and wherein the processing circuitry is configured to detect the one or more sleep apnea episodes of the patient occurring during the first period based on the determined heart rates by detecting the one or more sleep apnea episodes of the patient occurring during the first period based on the one or more heart rate parameters.

7. The medical device of claim 1, wherein the processing circuitry is configured to detect respirations based on the measured impedances of the patient, and wherein the processing circuitry is configured to detect the one or more sleep apnea episodes of the patient occurring during the second period based on the measured impedances by detecting the one or more sleep apnea episodes of the patient occurring during the second period based on the respirations.

8. The medical device of claim 1, wherein the processing circuitry is further configured to determine a heart rate variability value based on the heart rates, wherein the one or more verification conditions comprise the heart rate variability value being less than the heart rate variability threshold.

9. The medical device of claim 1, wherein the processing circuitry is further configured to, responsive to (1) detecting the one or more sleep apnea episodes of the patient occurring during the first period and (2) not detecting the one or more sleep apnea episodes of the patient occurring during the second period, discard from memory circuitry of the medical device indications of the one or more sleep apnea episodes of the patient occurring during the first period.

10. The method of claim 1, wherein the processing circuitry is further configured to, responsive to (1) detecting the one or more sleep apnea episodes of the patient occurring during the first period and (2) not detecting the one or more sleep apnea episodes of the patient occurring during the second period, generate an indication that the patient has experienced limb movement during the first period.

11. The medical device of claim 1, wherein the processing circuitry is further configured to stop detection of sleep apnea episodes based on the measured impedances in response to the second period lasting for a predetermined duration.

12. The medical device of claim 11, wherein the pre-determined duration is 2 minutes.

13. The medical device of claim 1, wherein the processing circuitry is further configured to, responsive to not detecting an atrial fibrillation episode based on the cardiac signal during the second period, stop detection of sleep apnea episodes based on the measured impedances.

14. A method comprising:
sensing, by sensing circuitry of a medical device, a cardiac signal indicating activity of a heart of a patient;
determining, by processing circuitry of the medical device, heart rates of the patient based on the cardiac signal sensed during a first period;
detecting, by the processing circuitry, one or more sleep apnea episodes of the patient occurring during the first period based on the heart rates;
determining, by the processing circuitry, whether one or more verification conditions are satisfied, wherein the one or more verification conditions comprise detection of a threshold number or a threshold rate of sleep apnea episodes occurring during the first period; and
responsive to determining that the one or more verification conditions are satisfied:
controlling, by the processing circuitry, the sensing circuitry to measure impedances of the patient during a second period subsequent to the first period; and
determining whether one or more sleep apnea episodes of the patient occurred during the second period based on the measured impedances.

15. The method of claim 14, further comprising detecting, by the processing circuitry, atrial fibrillation episodes based on the cardiac signal, wherein the one or more verification conditions comprise detection of an atrial fibrillation episode during the first period.

16. The method of claim 14, further comprising determining one or more heart rate parameters based on the heart rates of the patient, and wherein detecting the one or more sleep apnea episodes of the patient occurring during the first period based on the heart rates comprises detecting the one or more sleep apnea episodes of the patient occurring during the first period based on the one or more heart rate parameters.

17. The method of claim 14, further comprising detecting respirations based on the measured impedances of the patient, and wherein detecting the one or more sleep apnea episodes of the patient occurring during the second period based on the measured impedances comprises detecting the one or more sleep apnea episodes of the patient occurring during the second period based on the respirations.

18. The method of claim 14, further comprising:
determining, by the processing circuitry, a heart rate variability value based on the heart rates; and
comparing, by the processing circuitry, the heart rate variability value to a heart rate variability threshold, wherein the one or more verification conditions comprise the heart rate variability value being less than the heart rate variability threshold.

19. The method of claim 14, further comprising, responsive to (1) detecting the one or more sleep apnea episodes of the patient occurring during the first period and (2) not detecting the one or more sleep apnea episodes of the patient occurring during the second period, discarding, by the processing circuitry and from memory circuitry of the medical device, indications of the one or more sleep apnea episodes of the patient occurring during the first period.

20. The method of claim 14, further comprising, responsive to (1) detecting the one or more sleep apnea episodes of the patient occurring during the first period and (2) not detecting the one or more sleep apnea episodes of the patient occurring during the second period, generating, by the processing circuitry, an indication that the patient has experienced limb movement during the first period.
